(19) United States
(12) Patent Application Publication
(10) Pub. No.: US 2012/0288885 A1
(43) Pub. Date: Nov. 15, 2012

(54) METHOD AND KIT FOR MEASURING ENZYMATIC ACTIVITIES OF VARIOUS CYTOCHROME P450 MOLECULE SPECIES COMPREHENSIVELY AND WITH HIGH EFFICIENCY

(75) Inventors: Hiromasa Imaishi, Kobe-shi (JP); Kenichi Morigaki, Ikeda-shi (JP); Yoshiro Tatsu, Ikeda-shi (JP); Gang Chang, Ikeda-shi (JP)

(73) Assignees: NATIONAL INSTITUTE OF ADVANCED INDUSTRIAL SCIENCE AND TECHNOLGOY, Tokyo (JP); NATIONAL UNIVERSITY CORPORATION KOBE UNIVERSITY, Kobe-shi, Hyogo (JP)

(21) Appl. No.: 13/393,706

(22) PCT Filed: Aug. 27, 2010
(86) PCT No.: PCT/JP2010/064567

§ 371 Date: May 11, 2012

(30) Foreign Application Priority Data
Sep. 1, 2009 (JP) 2009-201187
Sep. 1, 2009 (JP) 2009-201190

(51) Int. Cl. C12Q 1/26; C12N 11/10

(52) U.S. Cl. 435/25; 435/178

(57) ABSTRACT

The present invention relates to a method, and a kit, for measuring the enzymatic activity of cytochrome P450 comprehensively and with high efficiency and accuracy, wherein an oxygen sensing layer and a cytochrome P450-supporting layer are vertically integrated on a chip, and cytochrome P450 is supported in a hydrophilic polymer matrix in the cytochrome P450-supporting layer,
the cytochrome P450 generates NADPH by being irradiated with light in the presence of at least one caged compound selected from the group consisting of caged-NADP and caged-G6P, an enzyme utilizing NADPH as a coenzyme (i.e., cytochrome P450 reductase) and a substrate thereof to supply NADP and/or G6P from the caged compound to generate NADPH to start the reaction between the enzyme and a substrate.

### TECHNICAL FIELD

This application claims the priority of Japanese patent application 2009-201187 and Japanese patent application 2009-201190 filed on Sep. 1, 2009, the entire contents of which are incorporated by reference herein.

The present invention relates to a technique for evaluating metabolic activities of P450 molecular species toward various chemical compounds with high efficiency. More specifically, the present invention relates to a vertically integrated chip comprising an immobilized cytochrome P450-supporting layer and an oxygen sensor, and the use thereof.

The present invention further relates to a method for measuring the enzymatic activities of NADPH dependent enzymes or oxidases reduced by the dependent enzymes, including cytochrome P450 reductase and cytochrome P450, and a kit used therefor. More specifically, the present invention relates to a kit for accurately measuring enzymatic activity by using UV illumination to supply NADPH, thus controlling the initiation of the enzymatic reaction.

### BACKGROUND ART

Cytochrome P450 relates to the detoxication metabolisms and metabolic activations of various chemical compounds, including agricultural chemicals and pharmaceuticals. Revealing the metabolic reactions due to P450 is important for evaluating the toxicity of xenobiotics (Non-Patent Literature 1). Recently, the application of P450 enzymes to the production of various substances or as an index for evaluating the safety of pharmaceuticals, agricultural chemicals, and the like has gained widespread attention (Non-Patent Literature 2). Particularly in the development of pharmaceuticals, the manifestation of toxicity by interaction between a compound and a P450 enzyme represents a major obstacle to new drug development. Therefore, the assessment of P450 enzyme metabolic activity toward new drug candidates is considered to be an important index in the initial stage of development. Further, it has recently become evident that the effects, side effects, and the like of a drug vary due to individual differences of P450 enzymatic activity attributable to genetic polymorphisms thereof. Therefore, a technique that can analyze the metabolic activity of various P450 molecular species including genetic polymorphisms toward a compound with high efficiency is in demand in fields such as personalized medical care (Non-Patent Literature 3).

Currently, 57 molecular species have been confirmed in the human cytochrome P450. Although each of the molecular species has individual difference in its enzymatic activity, it is reported that each molecular species is involved in the metabolism of various pharmaceutical compounds, benzene and other organic solvents, low molecular carcinogens in the environment, etc. (Non-Patent Literature 4).

Oxygen sensors using a fluorophorefluorophore (e.g., ruthenium complex) whose fluorescence intensity changes depending on the oxygen concentration are used in biosensors and bioassays. A product, by which a cell culture and enzymatic activity can be evaluated in parallel, by providing an oxygen sensing layer at the bottom of a multiwell plate is commercially available. Such a product is also used for evaluating the activity of P450 enzymes suspended in an aqueous solution (Non-Patent Literature 5-7).

In recent years, research and development is being increasingly conducted using micro reactors and biosensors in which miniscule flow channels and wells are produced by forming depressions and projections on quartz or silicone polymer (PDMS) by microprocessing technology (Non-Patent Literature 8).

When enzymatic activity is measured using miniscule microwells or flow channels, it is generally difficult to mix solutions in such a small space. Controlling the initiation of the enzymatic reaction thus becomes problematic. As a means for solving this problem, there is a technique for controlling the initiation of reaction using light. A molecule that is designed to have its activity suppressed by adding a photoremovable protecting group to a bioactive molecule and to have its bioactivity recovered by deprotection caused by UV illumination is referred to as a caged compound, and the caged compound is widely used as a tool for analyzing the mechanisms of biological molecules. The caged compound itself is inactive, and the active compound is liberated by the deprotection of the protecting group upon UV illumination. Caged compounds of NADP and G6P are known (Patent Literature 1, Non-Patent Literature 9).

### CITATION LIST

### Patent Literature

PTL 1: U.S. Pat. No. 6,020,480

### Non-Patent Literature

NPL 1: Chem Res Toxicol., 21, 70-83 (2008)
NPL 2: K. R. Korzekwa, and J. P. Jones, Pharmacogenetics 1993, 3, 1-18
NPL 3: J. van der Weid, L. S. Steijns, Ann Clin Biochem. 1999, 36, 722-9
NPL 4: Cancer Res., 47, 3378-3383 (1987)
NPL 5: S. M. Borisov and O. S. Wolfbeis, Chem. Rev. 2008, 108, 423-461
NPL 6: Z. Rosenzweig and R. Kopelman, Anal. Chem. 1996, 68, 1408-1413
NPL 7: X. Wu, M. M. F. Choi, D. Xiao, Analyst 2000, 125, 157-162
NPL 8: B. H. Weigl, R. L. Bardell, C. R. Cabrera, Adv. Drug Delivery Rev. 2003, 55, 349-377
NPL 9: R. R. Swezey, D. Epel, Exp. Cell Res. 1992, 201, 366-372

### SUMMARY OF INVENTION

### Technical Problem

An object of the present invention is to provide a technique for detecting the enzymatic activities of various P450 molecular species toward substrate molecules with high efficiency. More specifically, the present invention aims to measure the drug-metabolizing enzyme activity of cytochrome P450 toward various chemical compounds, particularly pharmaceuticals or foods, with higher efficiency and greater accuracy than known assay methods.

### Solution to Problem

The present invention relates to a technique for measuring the metabolic activities of P450 molecular species toward various chemical compounds in a comprehensive and highly efficient manner. In order to achieve this object, the measurement of enzymatic activity is performed by vertically integrating an oxygen sensing layer and immobilized cytochrome P450, and combining the result with microstructures such as micro-flow channels and microwells. Furthermore, by photoregulating the supply of coenzyme (NADPH) necessary for the enzymatic activity of cytochrome P450, the reactions of substrate solutions encapsulated in a large number of microwells can be simultaneously initiated by UV illumination. These techniques make it possible to simultaneously measure the initial velocity of the metabolic response of various P450 molecular species toward chemical compounds and to measure P450 metabolic activity with higher efficiency and accuracy than is possible with conventional assays.

The present inventors came up with the idea that enzymatic reaction assays for various P450 molecular species can be performed with high efficiency by vertically integrating a uniform silica layer (oxygen sensor) containing a ruthenium complex and cytochrome P450 immobilized in a matrix, and combining the result with a flow channel formed by a microprocessing technology as shown in FIG. 1.

The present inventors further realized that the activity of either cytochrome P450 reductase or cytochrome P450 can be regulated by supplying NADPH via an NADPH regenerating system, and showed that enzymatic activity can be photoregulated by adding a photoremovable protecting group, which is necessary for the NADPH regenerating system, to NADP and/or G6P (FIGS. 12 and 13).

The present invention provides a vertically integrated chip and the use thereof as described below, and methods or kits for measuring the enzymatic activity of an NADPH dependent enzyme as described below.

Item 1. A vertically integrated chip comprising an oxygen sensing layer and a cytochrome P450-supporting layer vertically integrated on a chip,

in the cytochrome P450-supporting layer, cytochrome P450 being supported in a hydrophilic polymer matrix.

Item 2. The vertically integrated chip according to Item 1, wherein the hydrophilic polymer is agarose gel.

Item 3. The vertically integrated chip according to Item 1, wherein the oxygen sensing layer contains a ruthenium complex in a silica matrix.

Item 4. The vertically integrated chip according to any one of Items 1 to 3, wherein the oxygen sensing layer and the cytochrome P450-supporting layer are vertically integrated in a micropore (microwell).

Item 5. The vertically integrated chip according to any one of Items 1 to 4, which further comprises a flow channel for introducing a substrate on the cytochrome P450-supporting layer.

Item 6. The vertically integrated chip according to Item 5, wherein the flow channel is a micro-flow channel.

Item 7. The vertically integrated chip according to any one of Items 1 to 6, wherein the oxygen sensing layer and the cytochrome P450-supporting layer are vertically integrated in the micro-flow channel in a uniform manner.

Item 8. The vertically integrated chip according to any one of Items 1 to 7, wherein the cytochrome P450-supporting layer comprises a plurality of cytochrome P450-supporting portions each having a cytochrome P450, and the vertically integrated chip is capable of analyzing metabolic activity of each cytochrome P450 toward a substrate.

Item 9. Use of the vertically integrated chip of any one of Items 1 to 8 to evaluate the oxidation reaction degree of cytochrome P450 toward a substrate.

Item 10. A method for identifying a compound comprising:

reacting a substrate with the vertically integrated chip of Item 8 to identify the compound based on metabolic patterns of a plurality of cytochrome P450s and substrates.

Item 11. A method for measuring enzymatic activity comprising:

irradiating light in the presence of at least one caged compound selected from the group consisting of caged-NADP and caged glucose-6-phosphate (G6P), an NADPH dependent enzyme, and, if necessary, an oxidase that is reduced by an NADPH dependent enzyme and a substrate thereof to generate NADPH by supplying NADP and/or G6P from said at least one caged compound to initiate a reaction of the NADPH dependent enzyme or oxidase with a substrate.

Item 12. The method according to Item 11, wherein the caged-NADP is represented by the following formula:

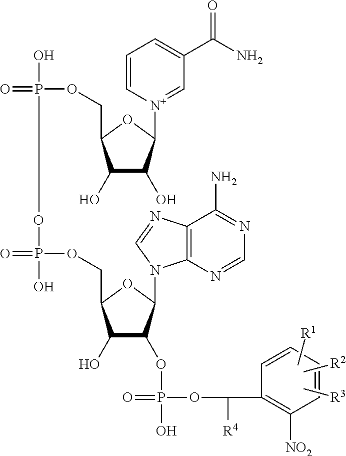

wherein R¹, R² and R³ may be the same or different and independently represent a hydrogen atom, a lower alkyl group, a lower alkoxy group, an amino group, a halogen atom, a hydroxy group or a cyano group; or any two of R¹, R² and R³ are combined to form a methylenedioxy group; and R⁴ represents a hydrogen atom or a methyl group.

Item 13. The method according to Item 11, wherein the caged-G6P is represented by the following formula:

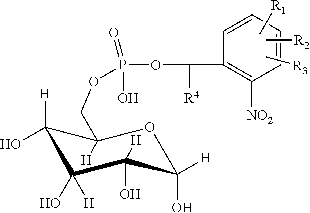

wherein R¹, R² and R³ may be the same or different and independently represent a hydrogen atom, a lower alkyl group, a lower alkoxy group, an amino group, a halogen atom, a hydroxy group or a cyano group; or any two of R¹, R² and R³ are combined to form a methylenedioxy group; and R⁴ represents a hydrogen atom or a methyl group.

Item 14. The method according to Item 11, wherein the NADPH dependent enzyme is a cytochrome P450 reductase.

Item 15. The method according to Item 11, wherein both the caged-NADP and the caged-G6P are made to coexist with an NADPH dependent enzyme and a substrate thereof.

Item 16. The method according to Item 15, wherein the NADPH dependent enzyme is a cytochrome P450 reductase.

Item 17. A kit comprising at least one caged compound selected from the group consisting of caged-NADP and caged-G6P, an NADPH dependent enzyme, and an oxidase that can be reduced by an NADPH dependent enzyme, the kit being used for measuring the enzymatic activity of the oxidase toward a substrate compound.)

Item 18. The kit according to Item 17, which comprises both the caged-NADP and the caged-G6P.

Item 19. The kit according to Item 17 or 18, wherein the NADPH dependent enzyme is a cytochrome P450 reductase.

Item 20. The kit according to Item 17 or 18, which comprises a microwell structure or a micro-flow channel, and which simultaneously activates multiple types of NADPH dependent enzymes by local or full-surface UV illumination to measure activities thereof in parallel.

Item 21. The kit according to Item 20, wherein the NADPH dependent enzyme is a cytochrome P450 reductase.

### Advantageous Effects of Invention

According to one embodiment of the present invention, by introducing a sample solution containing a compound that is a possible substrate for cytochrome P450 to the surface of a vertically integrated chip in which P450 is immobilized on an oxygen sensor, it is possible to quickly assay the degree to which the substrate is oxidized by P450. By immobilizing P450 on the surface of an oxygen sensor, the sensitivity of enzymatic activity detection can be increased remarkably. The oxidation reaction of P450 always involves oxygen consumption; therefore, the oxygen sensor can detect reactions of any P450 molecular species (the molecular species is not limited as it is with assays using a fluorogenic substrate). The use of immobilized P450 enables the compound-containing solution to be exchanged, so a plurality of reaction solutions can be sequentially supplied repeatedly. Furthermore, by combining the vertically integrated chip with micro-flow channels, assaying can be performed with very small amounts of reaction solution, and a plurality of samples can be simultaneously assayed. Immobilizing a plurality of P450s and using them to react with a substrate also makes it possible to identify the substrate.

According to another embodiment of the present invention, by photoregulating the supply of a coenzyme (NADPH) that is required for the enzymatic activity of cytochrome P450, the reactions of a substrate solution encapsulated in a plurality of microwells can be simultaneously initiated by UV illumination to simultaneously measure the initial metabolic reaction velocity of various P450 molecular species toward a chemical compound (FIG. 13). When the enzymatic activity is measured using microwells or micro-flow channels, it is generally difficult to mix solutions in such a small space, causing a problem for regulating the initiation timing of the enzymatic reaction. In the present invention, NADP and/or G6P are/is supplied into a reaction system by irradiating light to generate NADPH so that the initiation of the enzymatic reaction of the NADPH dependent enzyme can be temporally and spatially controlled. For example, by regulating the start of the reaction of cytochrome P450 to suitably select the initial velocity of the reaction, the metabolic capacity of the P450 enzyme toward various chemical compounds can be evaluated in a more quantitative manner. Furthermore, the enzymatic reactions of many samples with different enzyme molecular species, compounds, concentrations, and the like can be started simultaneously by UV illumination; therefore, high throughput due to mechanization can be achieved. Because caged-NADP is endogenous NADP, it exhibits a slight background reaction, but caged-G6P exhibits very little background reaction. Combining caged-NADP with caged-G6P enables higher photoregulation.

The effects described above make it possible to measure P450 metabolic activities with higher efficiency and accuracy than conventional assays.

### BRIEF DESCRIPTION OF DRAWINGS

FIG. 1 shows a vertically integrated chip of the present invention and an example of combining the vertically integrated chip with flow channels.

FIG. 2 illustrates the preparation of a ruthenium doped silica gel layer. It was found that the best fluorescence homogeneity is exhibited near TEOS:Octyl-triEOS=5:5.

FIG. 3 shows fluorescence responses of oxygen sensors due to the metabolic reaction of P450 (human CYP1A1)-containing membrane fractions encapsulated in different matrixes: (A) P450 encapsulated in agarose gel, (B) P450 encapsulated in Ludox gel, and (C) P450 encapsulated in silica gel. In the figures, the circles () indicate responses in the presence of the substrate (0.5 mM chlortoluron), and the squares (▪) indicate responses in an NADPH solution without the substrate.

FIG. 4: (A) shows change in the fluorescence responses (time course) of P450 (human CYP1A1) encapsulated in agarose gel toward different concentrations of chlortoluron. (B) shows the differential values (displacement rate) of increases in fluorescence intensity shown in FIG. 4A. (C) shows a correlation curve between the maximum values of the fluorescence displacement rate (Max. rate) and the concentration of chlortoluron.

FIG. 5 illustrates an example of the design of a micro-flow channel, wherein microwells (50 μm) are located at equal intervals in a 100-μm wide flow path (4 channels). In each well, an oxygen sensor and an enzyme-immobilized gel are vertically integrated.

FIG. 6 illustrates an example of a desigen of micro-flow channel, wherein microwells (50 μm) are located at equal intervals in a 100-μm wide flow path. In each well, an oxygen sensor and an enzyme-immobilized gel are vertically integrated.

FIG. 7 illustrates an example of the design of a micro-flow channel, wherein an oxygen sensor and enzyme-immobilized gel are vertically integrated in a predetermined position of a 100-μm wide flow path. As a solution proceeds in the flow path, the metabolism of a chemical compound toward P450 progresses, and the fluorescence intensity of the oxygen sensor increases (the reaction velocity can be assayed based on the spatial distribution of fluorescence intensity of the oxygen sensor).

FIG. 8 is a schematic illustration of P450 encapsulated in agarose gel vertically integrated on an oxygen sensor in a microwell. (1) Polymer cover; (2) Substrate solution (e.g. 7-EC, BP); (3) Agarose gel doped P450 microsome; and (4) Oxygen-sensing layer (Ru complex).

FIG. 9 shows a comparison of the response of an oxygen sensor due to the metabolism of CYP1A1-agricultural chemical (chlortoluron). In the figure, the squares (▪) indicate P450 encapsulated in agarose gel (vertically integrated structure) and the circles () indicate P450 suspended in a solution. By encapsulating P450 in agarose gel, the detection sensitivity increased to about 10 times.

FIG. 10-1 shows fluorescence responses of an oxygen sensor/immobilized P450 toward ingredients in food products and an agricultural chemical (chlortoluron) ((A) CYP1A1, (B) CYP2C8, (C) CYP2E1 and (D) CYP3A4), and the time course of the change in oxygen sensor fluorescence intensity.

FIG. 10-2 shows fluorescence responses of an oxygen sensor/immobilized P450 toward ingredients in food products and an agricultural chemical (chlortoluron) ((1) CYP1A1, (2) CYP2C8, (3) CYP2D6, (4) CYP2E1 and (5) CYP3A4), and the maximum values of the response of the oxygen sensor based on the activity of P450 molecular species toward each compound (the ratio to the measured value without a substrate (NADPH) was determined as the longitudinal axis). This indicates that the sensor can be used for identifying compounds by patternizing the fluorescence responses.

FIG. 11 shows an assay of the activity of various P450 molecular species toward capsaicin using an oxygen sensor/immobilized P450: normalized by the response toward a solution without a substrate (background oxygen consumption). This indicates that the sensor can be used for identifying compounds by patternizing the fluorescence responses. Each peak indicates, from the left, CYP2C9, CYP1A2, CYP2D6, CYP3A4, CYP2B6, CYP2C19 1A, CYP2C19 1B, CYP2E1, CYP1A1, CYP2C8, CYP2W1, CYP4X1, CYP17A1, CYP27A1, CYP51A1, CYP2A6, CYP2A13, CYP1B1, CYP2C18, CYP2J2, CYP3A5, CYP2R1, pcW and CYP2B6, wherein the peak of CYP3A5 is particularly high.

FIG. 12 is a conceptual diagram schematically illustrating the regulation of enzymatic activity using a caged coenzyme. The caged coenzyme (inactive) added to the reaction system is transformed to an active compound by UV illumination; therefore, the reaction can be “immediately” started with a predetermined timing and position. The advantages of using a caged coenzyme are: (i) the mechanical portion can be simplified, (ii) the enzyme and substrate can be mixed in advance, and (iii) it can be advantageously used in the initial analysis.

FIG. 13 is a conceptual diagram schematically illustrating the regulation of enzymatic activity by caging an NADPH regenerating system.

FIG. 14 shows the activation of cytochrome P450 by irradiating caged-NADP with UV light: the correlation between enzymatic activity toward a fluorogenic substrate of human CYP1A1 (7-ethoxyresorufin: 7-ER) and the UV light irradiation time. (A) only caged-NADP was illuminated, (B) P450 was illuminated (normal NADP was used), and (C) caged-NADP was illuminated in the presence of P450 (human CYP1A1).

FIG. 15 shows the activation of cytochrome P450 (human CYP1A1) by irradiating caged-G6P with UV light: the correlation between enzymatic activity of human CYP1A1 toward a fluorogenic substrate (7-ethoxyresorufin: 7-ER) and the UV light irradiation time. (A) only caged-G6P was illuminated, (B) P450 was illuminated (normal G6P was used), and (C) caged-G6p was illuminated in the presence of P450.

FIG. 16 shows the activation of cytochrome P450 by irradiating caged-NADP/caged-G6P with UV light in the presence of cytochrome P450 (human CYP1A1): the correlation between enzymatic activity of human CYP1A1 toward a fluorogenic substrate (7-ethoxyresorufin: 7-ER) and the UV light irradiation time.

FIG. 17 shows activation of cytochrome P450 by irradiating caged-NADP and/or caged-G6P singly or in combination with UV light in the presence of cytochrome P450 (human CYP1A1): correlation between enzymatic activity of human CYP1A1 toward fluorogenic substrate (7-ethoxyresorufin: 7-ER) and the UV light irradiation time. The horizontal axis indicates the UV light irradiation time, the squares (U) indicate the decaging of caged-G6P, the circles (*) indicate the decaging of caged-NADP, and the triangles (A) indicate the decaging of both caged-G6P and caged-NADP. The activity was normalized to values measured using normal G6P and NADP.

FIG. 18 shows enzyme activation of cytochrome P450 (human CYP1A1) by local irradiation of UV light: a reaction solution containing human CYP1A1, 7-ER and caged-G6P (natural NADP was used) was encapsulated in PDMS microwells, and only a single microwell (indicated by the arrow) was irradiated with UV light. Only the cytochrome P450 in the irradiated microwell exhibited enzymatic activity and fluorescence due to the metabolism of 7-ER was observed. (Left) Image observed using a bright field microscope. (Right) Image observed using a fluorescence microscope. Each microwell was 100 μm wide and 30 μm deep.

FIG. 19 shows enzyme activation of cytochrome P450 (human CYP1A1) by local UV light irradiation: a reaction solution containing human CYP1A1, 7-ER and caged-G6P (natural NADP was used) was encapsulated in PDMS microwells, and only a single microwell was irradiated with UV light. (A) shows the change of fluorescence intensity over time for the illuminated microwell (▪) and the adjacent microwells (). (B) shows fluorescence microscope images of illuminated microwells. Observation at the times shown in (A). Each microwell was 100 μm wide and 30 μm deep.

FIG. 20 shows activation of cytochrome P450 (human CYP1A1) enzyme by introducing substrates with different concentrations into micro-flow channels and irradiating them with UV light at the same time: a reaction solution containing human CYP1A1, 7-ER and caged-G6P (natural NADP was used) was introduced into PDMS micro-flow channels and all of the channels were irradiated with UV light at the same time. In all channels, cytochrome P450 attained enzymatic activity and fluorescence due to metabolism was observed according to the concentration of 7-ER. 7-ER=(a) 0.35 μM, (b) 0.69 μM, (c) 1.73 μM, (d) 3.45 μM. Each channel was 60 μm wide and 30 μm deep.

FIG. 21 shows P450's enzymatic reaction toward different substrate concentrations. Metabolic activity of human CYP1A1 toward a fluorogenic substrate (7-ER) was observed in microwells using a fluorescence microscope. When the reaction was started by decaging caged-G6P by UV light irradiation, increases in fluorescence depending on the substrate concentration were observed. Each microwell was 100 μm wide and 30 μm deep.

FIG. 22 shows metabolic activity of human CYP1A1 toward a fluorogenic substrate (7-ER). G6P and caged-G6P are compared in terms of (Left) Michaelis-Menten plots and (Right) reaction kinetic constant. In the assay using caged-G6P, error values in Kₘₐₓ and Vₘₐₓ are smaller than in those using normal G6P; therefore, a measurement with higher data accuracy became possible.

FIG. 23 shows the results of a competitive assay using a fluorogenic substrate (7-ER) and a non-fluorogenic substrate (benzopyrene): the effect of 7-ER on the initial reaction velocity was examined while changing the benzopyrene concentration, and the results showed that benzopyrene functioned as a noncompetitive inhibitor on 7-ER. Solid line: only 7ER, Broken line: benzopyrene (0.1 uM), Dotted-line: benzopyrene (1 uM).

FIG. 24 shows the detection of enzymatic activity using an oxygen sensor. It was confirmed that the enzymatic reaction could be started by encapsulating a reaction solution containing a fluorogenic substrate (7-ER), caged-G6P and other necessary reagents in microwells (Left) in which an oxygen sensor and immobilized P450 (human CYP1A1)/agarose gel are vertically integrated, and irradiating the reaction solution with UV light (Right). (I) sealing tape; (II) encapsulated substrate solution; (III) plastic material (PMMA); (IV) P450/gel; and (V) oxygen sensor.

### DESCRIPTION OF EMBODIMENTS

The present invention is divided into two categories below, i.e., the invention relating to a vertically integrated chip and the invention relating to a caged compound. The present invention is explained in detail below.

### (I) The First Invention (the Invention Relating to a Vertically Integrated Chip)

In the present specification, any type of P450 of all organism species including membrane-bound P450s of mammals, insects, plants, etc.; soluble P450s of microorganism, bacteria, etc.; and others can be used. Examples of mammals include humans, monkeys, cows, horses, pigs, sheep, mice, rats, rabbits, dogs and cats. Among these, human cytochrome P450 is particularly preferable. Currently, it is known that there are 57 human P450s, including the following:

CYP1A1, CYP1B1, CYP1A2, CYP2A6, CYP2B6, CYP2A13, CYP2B6, CYP2C8, CYP2C9, CYP2C18, CYP2C19(1A,1B), CYP2D6, CYP2E1, CYP2J2, CYP2R1, CYP2W1, CYP3A4, CYP3A5, CYP3A7, CYP4X1, CYP17A1, CYP27A1, CYP51A1. In the present invention, P450s may be immobilized singly or in a combination of two or more. In the case of membrane-bound P450s, it is necessary to supply, at the same time, a cytochrome P450 reductase for electron transfer.

Any materials, such as glass, plastic, metal and ceramics, can be used as the plate of the present invention.

An oxygen sensing layer may be formed on the chip.

The oxygen sensing layer comprises an oxygen sensor and a matrix. Examples of oxygen sensors include ruthenium complexes and platinum complexes. Among these, ruthenium complexes are preferable and Ru(dpp)₃Cl₂ is particularly preferable. Examples of matrixes include ceramics such as silica, alumina, zirconia and titania; and polymer materials such as polyvinyl alcohol (PVA). Among these, silica is preferable.

An oxygen sensor, such as a ruthenium complex, can be encapsulated in silica by a sol gel process. Such an oxygen sensor (a ruthenium complex) can be obtained by applying a silica precursor solution containing an oxygen sensor (a ruthenium complex) to the surface of the chip with a spin coat method and then drying the result.

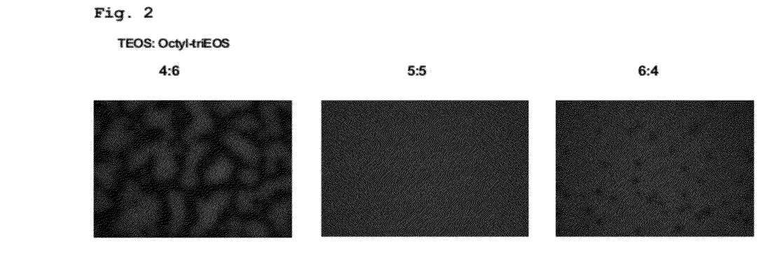

The sol gel process was optimized based on the processes reported in various documents so that the oxygen sensing layer exhibits uniform fluorescence intensity. It turned out that, among various aspects, the mixing ratio of the silica precursor (TEOS and OclyI-lriEOS) in the process of preparing silica gel imparted an important effect on the uniformity of the fluorescence intensity of the oxygen sensor (FIG. 2). The mixing ratio of TEOS:Oclyl-triEOS is most desirably 5:5; however, other mixing ratios may be employed as long as a change in the fluorescence can be detected, and any silica precursors may be used.

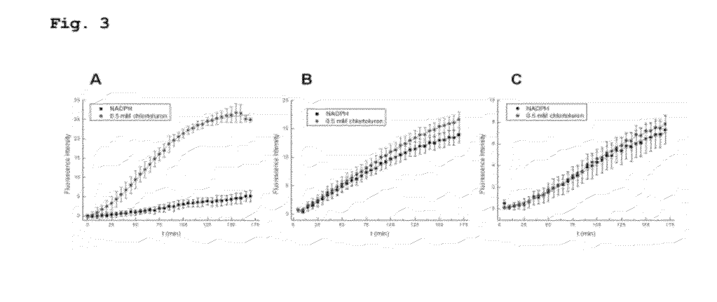

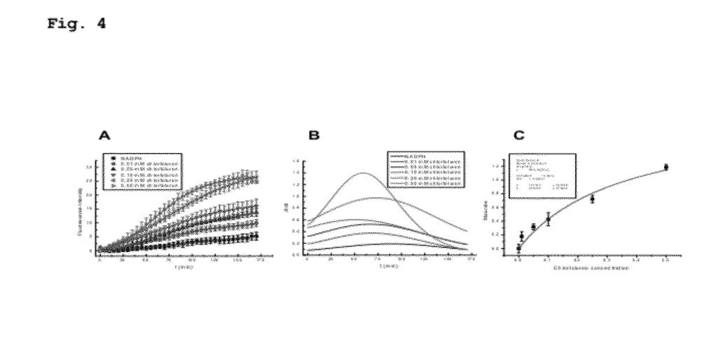

Subsequently, the P450 is preferably immobilized in a matrix of a hydrophilic polymer. Examples of hydrophilic polymers include cellulose derivatives such as polyvinyl alcohol (PVA), hydroxypropylmethylcellulose (HPMC), sodium carboxymethylcellose (CMC-Na) and hydroxyethylcellulose (HEC); polysaccharides such as alginic acid, hyaluronic acid, agarose, starch, dextran and pullulan, and derivatives thereof; homopolymers such as carboxy vinyl polymer, polyethylene oxide, poly(meth)acrylamide and poly(meth)acrylic acid; copolymers or mixtures of these homopolymers and polysaccharide, etc.; copolymers of other monomers; and polyion complex membranes of alginic acid or like polyanion with poly-L-lysine or like polycation. A preferable example is agarose gel. Because P450 (e.g., human CYP1A1) immobilized in agarose gel has a high enzymatic activity, it is preferably used to detect the oxygen consumption attributable to the immobilized enzyme reaction using an oxygen sensor (FIG. 3). In contrast, the P450 immobilized in silica gel exhibited very little increase in the amount of oxygen consumption compared to the background oxygen consumption even in the presence of a substrate. In the case of human CYP1A1 immobilized in agarose gel, a change in the oxygen consumption amount was observed by changing the concentration of the model compound (chlortoluron: herbicide) (FIG. 4). As a result of plotting the maximum values of increased velocity of fluorescence versus the substrate concentration, a concentration dependency that can be approximately fitted to the Michaelis-Menten kinetics was found.

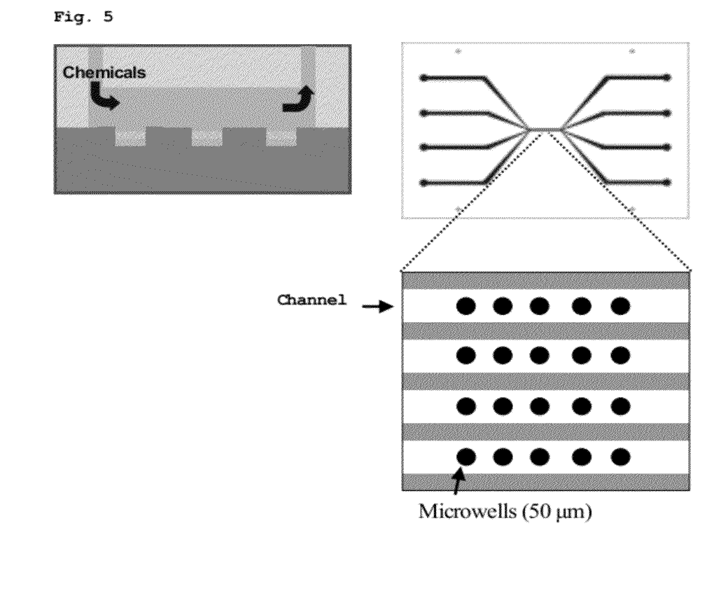

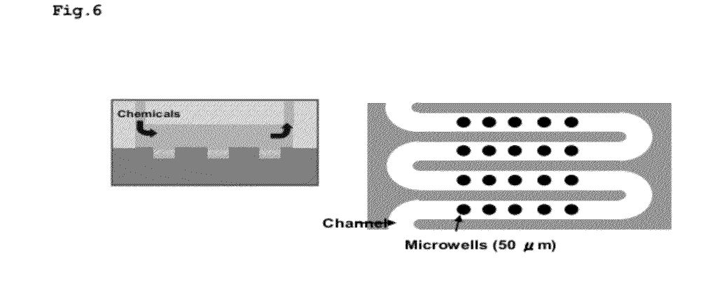

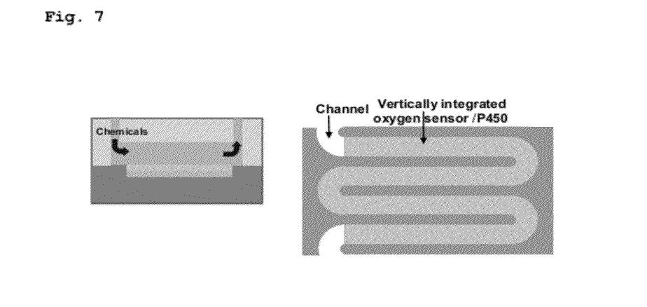

The aforementioned oxygen sensor and immobilized P450 can be utilized in a form incorporated in a microstructure (e.g., a microwell, a micro-flow channel or a combination thereof) formed of a silicone elastomer resin (poly-dimethylsiloxane (PDMS)), a photocurable resin, quartz glass, and the like. Preferable embodiments include the microwells and micro-flow channel designs shown in FIGS. 5 to 7.

From the above results, it is presumed that by combining P450 immobilized on an oxygen sensor with a micro-flow channel, the enzymatic activities of a large number of human P450 molecular species in various samples toward various chemical compounds can be assayed quickly in a parallel manner, as shown in FIGS. 5 to 7.

The main features of the present invention are as follows.

(1) The oxygen sensor can detect the activities of any P450 molecular species including genetic polymorphisms (the (molecular species is not limited as it is with assays using a fluorogenic substrate).

(2) By using immobilized P450, the solution including a compound can be replaced; therefore, a plurality of reaction solutions can be sequentially supplied repeatedly.

(3) By combining the vertically integrated chip with micro-flow channels, assays can be performed with very small amounts of reaction solution.

(4) A plurality of samples can be simultaneously assayed.

### (II) The Second Invention (the Invention Relating to a Caged Compound)

In the present invention, examples of enzymes whose enzymatic activities are measured include NADPH-dependent enzymes, or arbitrary oxidoreductases including NADPH-dependent enzymes that play a part in a series of oxidation-reduction reactions, such as enzymes that can be reduced by an NADPH dependent enzyme, in particular, oxidases. As such an oxidase, cytochrome P450 is preferably exemplified. As an NADPH dependent enzyme, cytochrome P450 reductase can be mentioned.

A molecule that is designed to have its activity suppressed by adding a photoremovable protecting group to a bioactive molecule and to have its bioactivity recovered by decaging caused by UV illumination is referred to as a caged compound, and the caged compound is widely used as a tool for analyzing the mechanism of a biomolecule. The inventors of the present invention focused, as the object caged compound, on an NADPH regenerating system that generates NADPH from NADP. The NADPH regenerating system generates NADPH from NADP by using glucose-6-phosphate (G6P) and glucose-6-phosphate dehydrogenase. Therefore, by using caged-NADP and/or caged-G6P that is obtained by adding a protecting group to NADP or G6P, the supply of NADPH that is necessary for the P450 enzymatic reaction can be photoregulated.

Note that, in the present invention, the activity of the NADPH dependent enzyme can be measured using caged-NADP and/or caged-G6P. Examples of known NADPH dependent enzymes include cytochrome P450 reductase, thioredoxin reductase, glutathione reductase, and NADPH-quinone reductase (NADPH QR), which is used for screening and identifying potential anticancer agents.

Among the above, cytochrome P450 reductase is coupled with the activity of cytochrome P450. Therefore, it becomes possible to assess the activity of P450 toward chemical compounds contained in various pharmaceuticals and foodstuffs by regulating the activity of cytochrome P450 reductase. Examples of known P450s include CYP1A1, CYP1B1, CYP1A2, CYP2A6, CYP2B6, CYP2A13, CYP2B6, CYP2C8, CYP2C9, CYP2C18, CYP2C19(1A,1B), CYP2D6, CYP2E1, CYP2J2, CYP2R1, CYP2W1, CYP3A4, CYP3A5, CYP3A7, CYP4X1, CYP17A1, CYP27A1 and CYP51A1. According to the present invention, the enzymatic activities of these P450s can be accurately measured.

The caged compound of the present invention is obtained by introducing a protecting group represented by Formula (I) or (IA) to NADP or G6P:

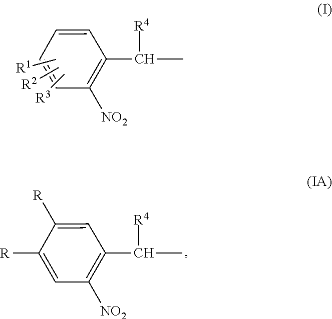

wherein R¹, R² and R³ may be the same or different and independently represent a hydrogen atom, a lower alkyl group, a lower alkoxy group, an amino group, a halogen atom, a hydroxy group or a cyano group; or any two of R¹, R² and R³ are combined to form a methylenedioxy group; and R⁴ represents a hydrogen atom or a methyl group. The locations to which the protecting group is introduced are shown below.

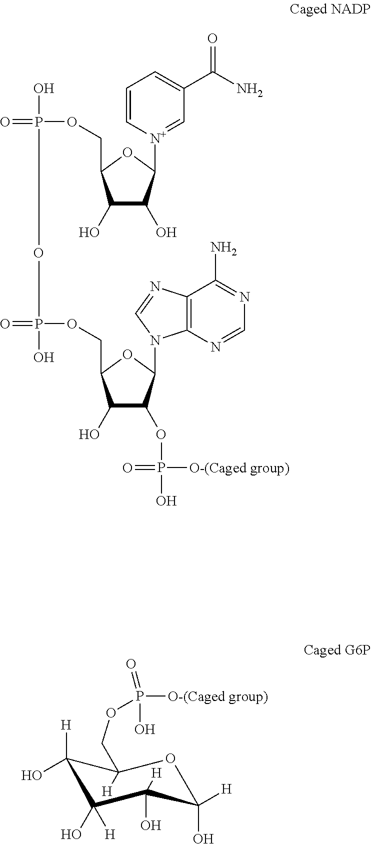

Examples of the lower alkyl groups represented by R¹, R² or R³ in Formula (I) include C₁₋₄ linear or branched alkyl groups, such as methyl, ethyl, n-propyl, isopropyl, n-butyl, isobutyl, sec-butyl and tert-butyl.

Examples of the lower alkoxy groups include C₁₋₄ linear or branched alkoxy groups, such as methoxy, ethoxy, n-propoxy, isopropoxy, n-butoxy, isobutoxy, sec-butoxy and tert-butoxy.

Examples of the halogen atoms include fluorine, chlorine, bromine and iodine.

A preferable group represented by Formula (I) is one in which any two of R¹, R² and R³ are hydrogen atoms and the remaining one is a hydrogen atom, a lower alkyl group or a lower alkoxy group, and R⁴ is a hydrogen atom.

The protecting group can be removed by UV light irradiation. There is no limitation to the UV light irradiated as long as it can remove a photosensitive group, and ordinary UV lamps, such as an Xe—Hg lamp (365 nm), can be used. The conditions for UV light irradiation are not particularly limited. For example, UV light can be irradiated by using a UV hand lamp for TLC detection (PU-2; manufactured by Topcon Corporation) for about 1 hour.

The kit of the present invention comprises at least one caged compound selected from the group consisting of caged-NADP and caged-G6P, an NADPH dependent enzyme, and, if necessary, oxidase that is reduced by an NADPH dependent enzyme, and the kit may further comprise a buffer solution of an NADPH dependent enzyme, a model substrate, etc. Furthermore, when the kit is used to assay P450 activity, it comprises at least one species of P450 in addition to a P450 reductase. The P450 activity can be assayed, for example, using the following model substrates.

TABLE 1

| P450 | Model substrate |
|---|---|
| CYP1A1, CYP1A2, | 7-ethoxyresorufin (7-ER), |
| CYP1B1 | 7-Ethoxycumarin (7EC) |
| CYP2A6 | Cumarin |
| CYP2B6 | 7-Ethoxy-4-trifluoromethylcoumarin |
| CYP2C8 | Chloromethyl fluorescin diethyl ether |
| CYP2C9 | Diclofenac |
| CYP2C18 | S-Mephenytoin |
| CYP2C19 | S-Mephenytoin |
| CYP2D6 | Bufuralol |
| CYP2E1 | Chlorzoxazone |
| CYP2J2 | Arachidonic acid |
| CYP3A4 | Testosterone |

P450 activities other than those described above and NADPH dependent enzymes other than P450 can be accurately assayed using a model substrate for each enzyme.

The caged compound of the present invention may be a known one or can be easily synthesized by a procedure disclosed in a known document, a procedure disclosed in Examples or a procedure according thereto.

### EXAMPLES

Preferable embodiments are explained in detail below with reference to the drawings.

### Production Example 1A

### Stable Expression of Human P450 Enzyme Protein in E. Coli, Preparation of P450-Containing Membrane Fractions and Evaluation of Activity

### 1. Expression of Human P450

Using a cassette plasmid for expressing P450, in which major human P450 genes (such as CYP1A1) and human NADPH-P450 reductase P450 were inserted in tandem with pCWRm1A2N, expression of P450 in E. coli was attempted. The transformation of E. coli was performed through the transformation of competent DH5α by a conventional method. Confirmation of the introduction of each plasmid into E. coli was conducted by evaluating drug resistance by means of antibiotic ampicillin added to an LB medium. A culture of recombinant E. coli was initiated by inoculating a single E. coli colony on an LB agar medium that contained the antibiotic ampicillin to 2.5 mL of TB liquid medium. Pre-culturing was performed at 37° C. for 16 hours. Subsequently, culturing was performed in an LB medium containing aminolevulinic acid having a final concentration of 500 μg/mL and ampicillin having a final concentration of 50 μg/mL for about 3 hours until the OD value became around 0.3. Upon lowering the temperature of the culture after culturing from 37° C. to 28° C., IPTG with a final concentration of 1 mM was added thereto and culturing was continued for 24 hours. The recombinant E. coli strains were collected from the E. coli liquid culture by centrifugation. The expression amount of each P450 enzyme protein in E. coli was evaluated by measurement with a reduced-CO difference spectrum. The reduced-CO difference spectrum was measured based on a conventional method by supplying CO under a reducing condition. The number of moles of P450 was calculated using the constant defined by Sato, Omura, et al. (T. Omura and R. Sato, J. Biol. Chem. 1964, 239, 2370-2378.).

### 2. Purification of Membrane Fractions

E. coli membrane fractions (microsomes) were purified in the following manner. 200 mL of a TB culture medium was centrifuged at 3,000 g for 10 minutes to harvest. Thereafter, ultrasonic fragmentation was conducted 6 times each for 30 seconds to fragment the cells. Subsequently, a liquid containing the resulting cell fragments was centrifuged at 10,000 rpm for 10 minutes to separate the residues in E. coli by centrifugation. The supernatant obtained after centrifugation was subjected to ultracentrifugation at 4° C. and 40,000 rpm (100,000 g) to collect membrane fractions containing P450 enzyme protein. Thereafter, the E. coli membrane fractions were dispersed in 3 mL of P450 storage buffer solution (100 mM potassium phosphate buffer (pH 7.5) containing 20% glycerol).

### 3. Activity Measurement

Drug metabolic activities of human CYP1A1 in the prepared recombinant E. coli were analyzed using high-performance liquid chromatography (HPLC). As the enzyme substrate, 7-ethoxycumarin, which is a P450 model fluorogenic substrate, was used. The following two enzymatic reaction methods were studied. One method directly added a substrate to a recombinant E. coli strain in which a previously cultured P450 enzyme protein was expressed. The other method used E. coli membrane fractions obtained by purifying a recombinant E. coli strain, in which a P450 enzyme protein was expressed, using an ultracentrifugal method. The oxidation reaction of P450, in the case where a P450 expressed E. coli strain was used, was conducted by adding various enzyme substrates in such a manner that each had a final concentration of 0.1 mM, and then incubating them at 28° C. for 50 hours. In the metabolism experiment using E. coli membrane fractions in which P450 was expressed, NADPH with a final concentration of 0.2 mM was added to the reaction solution as a coenzyme. The HPLC analysis was conducted using the D7000 HPLC System (manufactured by Hitachi Ltd.) with a C18 reverse phase column (COSMOCIL (5C18-AR), manufactured by Nacalai Tesque Inc.), and employing a linear gradient elution method using an eluent of MeOH/H₂O (containing 0.85% phosphoric acid) with a ratio of 35:65 to 100:0.

### Example 1A

### 1. Materials

Tetraethyl orthosilicate (TEOS), triethoxy (octyl) silane (Octyl-triEOS), Ludox HS-40 colloidal silica, agarose (Type VII), and sodium silicate solution were purchased from Sigma-Aldrich. Tris(4,7-diphenyl-,10-phenanthroline) ruthenium dichloride (Ru(dpp)₃Cl₂), ethanol, methanol, and concentrated hydrochloric acid were obtained from Wako Pure Chemical Industries. Potassium dihydrogen phosphate, β-nicotinamide adenine dinucleotide phosphate tetrasodium salt (NADPH), and dipotassium hydrogen phosphate were purchased from Nacalai Tesque. Chlortoluron was obtained from Riedel-de Haen. Glucose-6-phosphate (G6P) was purchased from Tokyo Chemical Industry. Glucose-6-phosphate dehydrogenase (G6PD) was purchased from Toyobo. Ninety-six microwell plates were purchased from Nunc. Milli-Q water with a resistivity of more than 18 MΩ·cm was used to prepare aqueous solutions. All chemicals and solvents were reagent grade and were used without further purification.

### 2. Instrumentation

All of the luminescence measurements were performed on a Fluoroskan Ascent CF (Labsystem) microplate reader controlled by Ascent software version 2.4 with excitation and emission wavelengths of 400 and 620 nm, respectively.

The measurements were conducted from the top of the wells (top mode) due to the transparency of agarose gel.

### 3. Preparation of Oxygen Sensing Layer on Microplate.

A ruthenium complex (Ru(dpp)₃Cl₂) doped sol solution was prepared as described in Anal. Chem. 75 (2003) 2407-2413 with small modifications as follows. TEOS (0.29 mL) was mixed with 0.612 mL of octyl-triEOS, 0.625 mL of ethanol, and 0.2 mL of 0.1 M HCl by stirring for 1 hour at room temperature. Then, 1.725 mL of ethanol was added to the solution to dilute the sol in order to improve the quality of the oxygen sensing film to be ultimately formed. The solution was further stirred for 1 hour. To prepare an Ru(dpp)₃Cl₂ doped sol, 100 μL of 2 mM Ru(dpp)₃Cl₂ in ethanol was mixed with 300 μL of the above-mentioned sol solution. This solution was capped and stirred for 30 min, and 10 μL thereof was pipetted into each well of the microplate. The microplate was stored in the dark at room temperature for gelling and aging for 6 days. In order to increase the hydrophilicity of the surface of the oxygen sensor to improve the attachment with hydrogel, the surface of the microarrays was modified with poly (vinyl acetate) (PVAC).

### 4. Encapsulation of P450-Containing Membrane Fractions in Agarose Gel, TEOS Gel, and Ludox Gel.

Agarose was dissolved in deionized water to form a 1.3% (w/w) solution at 60° C. This solution was cooled to about 38° C. A P450 suspension (100 μL) was mixed with 300 μL of 1.3% agarose sol, then 60 μL of a mixture of P450 and agarose sol was pipetted onto the surface of the oxygen sensing layer in each well of the microplate. The microarrays were kept in a refrigerator at 4° C. until use. A schematic illustration of the P450 encapsulated in agarose gel in the oxygen sensing microarrays is shown in FIG. 8.

TEOS sol was prepared by mixing 0.5 mL of TEOS, 0.25 mL of deionized water, and 12.5 μL of 0.1 M HCl and stirring for 3 hours to form a homogeneous sol. The sol was diluted four times with deionized water. Diluted TEOS sol (300 μL) was mixed with 100 μL of a P450 microsome suspension, and 60 μL of the mixed solution was pipetted onto the surface of the oxygen sensing layer in each well of the microplate. The microplate was stored in a refrigerator at 4° C.

A Ludox sol was prepared as described in the literature ((Anal. Chem. 77 (2005) 7080-7083, and J. Mater. Chem. 13 (2003) 203-208). More specifically, 0.5 mL of 8.5 M Ludox colloidal silica was mixed with 0.5 mL of 0.16 M sodium silicate solution while stirring. HCl (4.0 M) was added to neutralize the pH value to around 7, then 100 μL of P450 microsome suspension was mixed with 300 μL of the above Ludox silica sol. One drop of P450 doped sol (60 μL) was added to each well of the microplate. The microplate was stored in a refrigerator at 4° C. before use.

### 5. Measurement of Substrate Metabolic Activities Using Immobilized P450/Oxygen Sensor Vertically Integrated Chip

In order to measure the substrate metabolic activities using an immobilized P450/oxygen sensor vertically integrated chip (i.e., a chip comprising an immobilized P450 and an oxygen sensor vertically integrated therein), a study was conducted using immobilized human CYP1A1 as the P450 and chlortoluron (a herbicide) as a substrate. Standard substrate solutions with various chlortoluron concentrations were prepared as follows. 25 μL of chlortoluron/ethanol solutions with different concentrations of chlortoluron (0.8, 4, 8, 20, 40 mM) were added to 1,975 μL of 0.1 mM KPB solution containing a NADPH regenerating system (0.1 mM NADPH, 3 mM MgCl₂, 3 mM G6P, and 0.4 U/mL G6P). The final concentrations of the chlortoluron were 0.01, 0.05, 0.1, 0.25, and 0.5 mM, respectively. A 250-μL portion of the standard solution with different concentrations of substrate was added to each well of the microplates containing an immobilized P450/oxygen sensor vertically integrated chip. A transparent polymer tape was used to seal the plate and prevent the oxygen in the air from mixing into the enzymatic reaction. After the addition of the substrate solution into the microarrays, the microplate was quickly placed onto the platform of a microplate reader for the fluorescence measurement. Fluorescence intensity was recorded every 5 min for 3 hours.

### Results

### 1. Metabolic Responses of Chlortoluron Due to P450 Encapsulated in Agarose Gel, Ludox Silica Gel and TEOS Silica Gel

Agarose gel, Ludox silica gel and TEOS silica gel were used as the matrixes for encapsulating P450, and metabolic activities were analyzed. FIG. 3A shows the change in fluorescence intensity of the oxygen sensing layer with time when a chlortoluron solution (0.5 mM) or a solution without chlortoluron (both contained an NADPH regenerating system) was introduced to P450 encapsulated in agarose gel. A small increase of fluorescence intensity was also observed without addition of the substrate (▪), which is attributed to a background reaction from NADPH oxidation in the presence of P450 enzyme. In the presence of a substrate (0.5 mM chlortoluron), the fluorescence intensity was significantly increased () and reached a steady state with the lapse of time. An increase in fluorescence intensity indicates that the P450 microsome encapsulated in agarose gel maintains P450 enzymatic activity as in the case where the P450 microsome is contained in an aqueous solution, and consumes oxygen due to the metabolic reaction toward chlortoluron. The change in fluorescence intensity indicates a kinetic behavior similar to that observed in a metabolic reaction of liberated P450 in a solution phase system. This is probably attributable to the fact that the micropore structure of agarose gel allows the supply of NADPH and the substrate by rapid diffusion.

FIGS. 3B and 3C show the fluorescence responses of P450 encapsulated in Ludox silica gel and P450 encapsulated in TEOS silica gel respectively vertically integrated onto oxygen sensors in the presence and absence of the substrate (0.5 mM chlortoluron). In FIG. 3B (indicated by squares ▪), higher background oxygen consumption from NADPH was observed in Ludox silica gel, compared with the results of P450 encapsulated in agarose gel. However, the fluorescence showed only a limited increase even with the addition of the substrate. This may be due to various reasons, e.g., P450 metabolic activity is suppressed in the inorganic Ludox silica gel, the substrate diffusion is restricted, and so on. P450 encapsulated in TEOS silica gel showed low background oxygen consumption in the absence of a substrate; however, no significant fluorescence increase was observed even in the presence of a substrate (FIG. 3C). This is probably because ethanol produced during the hydrolysis of TEOS lowered the P450 enzymatic activity.

In order to evaluate the stability of P450 microsomes in agarose gel, P450 metabolism microarrays were kept for 10 days and 21 days and the P450 microsome activity was evaluated using the same method as that employed in chlortoluron experiments. P450-containing microarrays exhibited similar catalytic behavior even after being kept for 3 weeks. This indicates that P450 activity is maintained for a long time by agarose gel encapsulation.

2. P450 Encapsulated in Agarose Gel Responses toward Various Substrate Concentrations
Chlortoluron solutions with different concentrations were introduced into P450 encapsulated in agarose gel, and the fluorescence responses of the oxygen sensors were evaluated. FIG. 4A shows the change of fluorescence intensity in time in the presence of chlortoluron solutions of different concentrations. P450 encapsulated in agarose gel is sensitive to changes in the concentration of the substrate and exhibited different levels of fluorescence intensity at different concentrations (FIG. 4A). It was observed that changes in fluorescence intensity with the lapse of time could be fitted to sigmoidal curves, with a high correlation coefficient of 0.99. This is similar to the behaviors observed in microbial biochemical oxygen demand biosensors (BOD). This data can be analyzed by a dynamic transient method (DTM) using differential values of fluorescence intensity. FIG. 4B shows the differential value (displacement rate) of the increase in fluorescence intensity shown in FIG. 4A. The displacement rate of fluorescence intensity increased for the first hour due to oxygen consumption resulting from the metabolization of the substrate by P450. Subsequently, the displacement rate decreased due to the exhaustion of the substrate or oxygen with time. FIG. 4C is a graph in which the maximum values of fluorescence displacement rate were plotted against the substrate (chlortoluron) concentration. The error bar indicates the standard deviation. The red curves were obtained by fitting the data to the Michaelis-Menten's equation. This indicates that the maximum values of fluorescence displacement rate obtained by the DTM method can be approximately evaluated using the Michaelis-Menten rate model.

### Example 2A

### Vertically Integrated Structure and Comparison of P450 Enzymatic Activity Detection in Solution

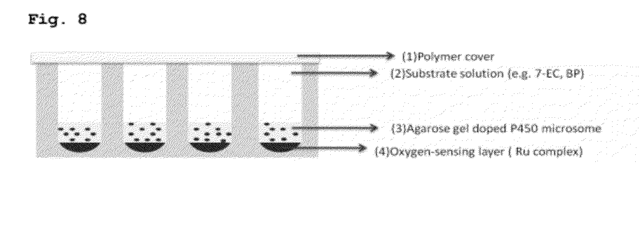

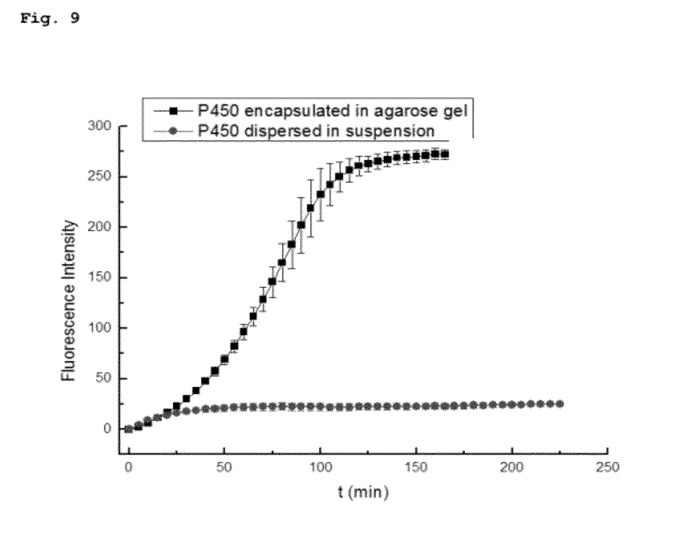

Using CYP1A1 as the P450 and chlortoluron as the substrate, a vertically integrated chip in which CYP1A1 was immobilized in agarose gel was produced in the same manner as in Example 1A, and the enzymatic activity of CYP1A1 was measured based on the fluorescence intensity. CYP1A1 was suspended in a solution with the same concentration (15 μL of membrane fraction sample was added), chlortoluron with a concentration of 0.2 mM was introduced, and the enzymatic activity of CYP1A1 was measured based on the change in the fluorescence intensity. FIG. 9 shows the results.

### Example 3A

### Metabolic Activities of P450 Molecular Species Toward Different Compounds

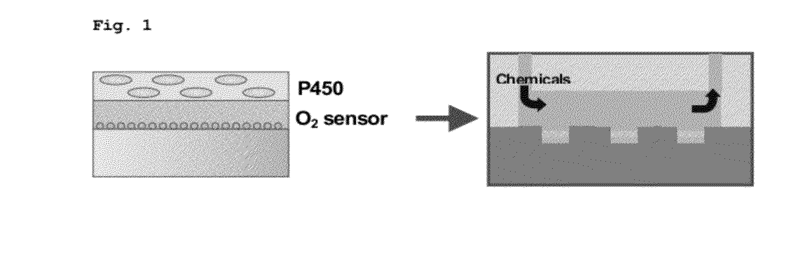

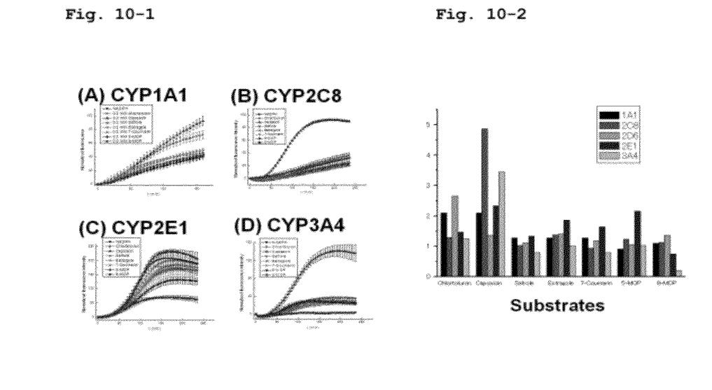

Using a 96-well microplate, human P450 of different molecular species ((A) CYP1A1, (B) CYP2C8, (C) CYP2E1, (D) CYP3A4) were immobilized on the surface of an oxygen sensor, and their fluorescence responses toward ingredients in food products (capsaicin, safrole, estragole, 7-Cumarin, 5-MOP, and 8-MOP) and an agricultural chemical (chlortoluron) were obtained. FIG. 10-1 and FIG. 10-2 show the results. FIG. 10-1 shows the change in the oxygen sensor fluorescence intensity time course. FIG. 10-2 shows the maximum value of the response of human P450 toward each compound. The longitudinal axis of FIG. 10-2 indicates the value obtained by dividing the value of the response with a substrate by the value of the response without a substrate (NADPH; background oxygen consumption) and standardizing the obtained value. As is clear from the figure, various molecular species show activity toward each compound. This result indicates that the sensor of the present invention can be used for identifying compounds by obtaining and patternizing the fluorescence responses of the sensor toward various compounds. This also indicates the possibility that activities of human P450 toward pharmaceutical compounds and like compounds can be detected in a parallel manner.

### Example 4A

### Metabolic Activities of Various P450 Molecular Species Toward a Compound

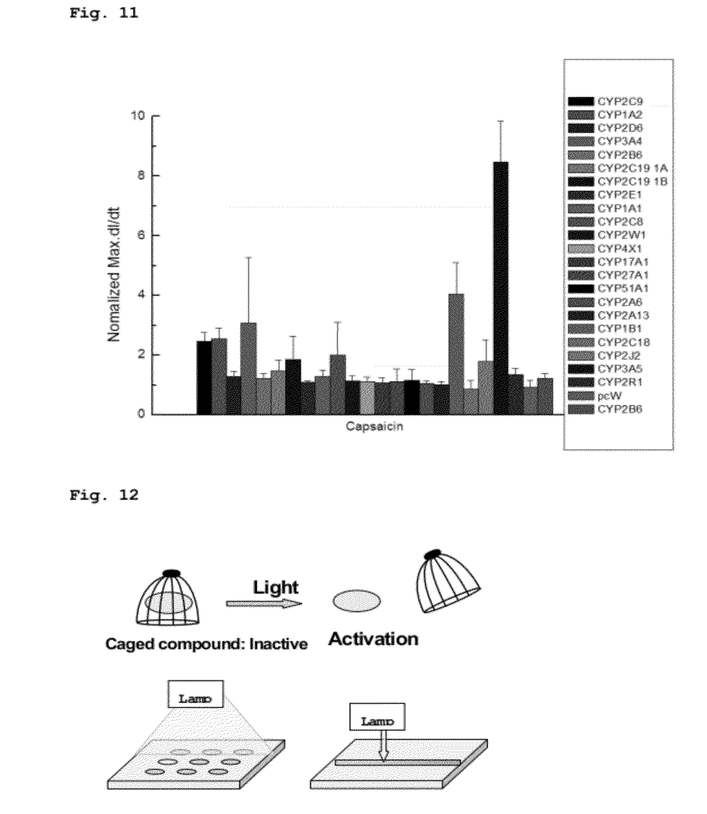

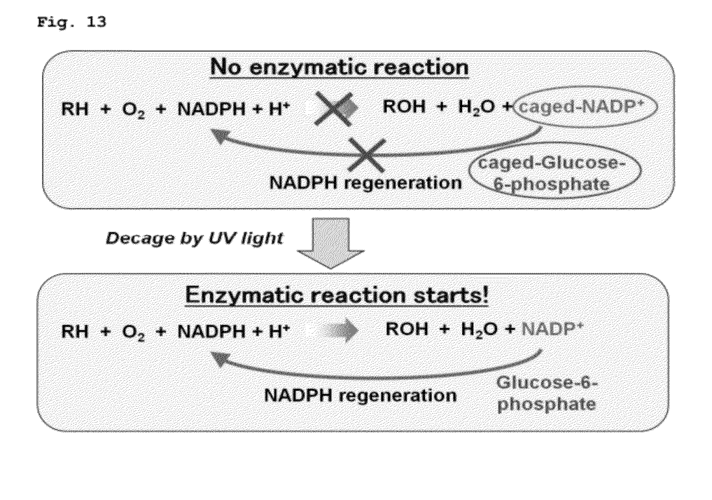

Using a 96-well microplate, the activity of various human P450 molecular species in oxygen sensor/immobilized P450 toward capsaicin was evaluated. The response to a solution with a substrate was compared to the response to a solution without a substrate and the activity of each molecular species was standardized. CYP2C9, CYP1A2, CYP2D6, CYP3A4, CYP2B6, CYP2C19(1A,1B), CYP2E1, CYP1A1, CYP2C8, CYP2W1, CYP4X1, CYP17A1, CYP27A1, CYP51A1, CYP2A6, CYP2A13, CYP1B1, CYP2C18, CYP2J2, CYP3A5, CYP2R1 and CYP2B6 were used as the P450. Furthermore, a membrane fraction (pCW) derived from E. coli was used as a negative control without human P450. FIG. 11 shows the results. The longitudinal axis of FIG. 11 indicates the value obtained by dividing the value of the response with a substrate by the value of the response without a substrate (background oxygen consumption) and standardizing the obtained value. As is clear from the figure, various molecular species show activity toward each compound. This result indicates that the sensor of the present invention can be used for identifying compounds by obtaining and patternizing the fluorescence responses of the sensor toward various compounds. This also indicates the possibility that activities of human P450 toward pharmaceutical compounds and like compounds can be detected in a parallel manner.

### Production Example 1B

### Synthesis of Caged-NADP

2-Nitrophenyl-acetophenone hydrazone (26.9 mg, 0.15 mmol) was dissolved in dichloromethane (0.3 mL), and manganese oxide (65.2 mg, 0.75 mmol) was added thereto. After being stirred for 5 minutes, the solution was centrifuged. The supernatant was filtered with a PTFE filter (manufactured by Millipore Corporation, pore diameter of 0.75 μm), and an NADP aqueous solution (obtained by dissolving 77 mg (0.1 mmol) of NADP in 0.3 mL of water) was added thereto, followed by stirring for 2 hours. The aqueous phase was washed twice with dichloromethane and freeze-dried to give 116 mg of white powder. The resulting white powder was purified by C18 reverse phase HPLC using an eluate containing acetonitrile and trifluoroacetic acid, and freeze-dried to give the target white powder (caged-NADP wherein the protecting group is represented by Formula I (R₁, R₂, R₃═H, R═CH₃)). Mass spectrometry (ESI): literature value of 892.4, obtained value of 893.1 for [M+H⁺]

### Production Example 2B

### Synthesis of Caged-G6P

2-Nitrophenyl-acetophenone hydrazone (1.26 mmol, 225 mg) was dissolved in dichloromethane (1 mL), and manganese oxide (369.9 mg) was added. After being stirred for 30 minutes, the solution was centrifuged. The supernatant was filtered with a PTFE filter (manufactured by Millipore Corporation, pore diameter of 0.75 μm), and a glucose-6-phosphate sodium salt aqueous solution (obtained by dissolving 87.3 mg (0.31 mmol) of glucose-6-phosphate sodium salt in 1 mL of water) was added thereto, followed by stirring overnight. The aqueous phase was washed twice with dichloromethane and freeze-dried to give 116 mg of white powder. The resulting white powder was purified by C18 reverse phase HPLC using an eluate containing acetonitrile and 10 mM ammonium bicarbonate, and freeze-dried twice to give the target white powder (caged-G6P wherein the protecting group is represented by Formula I (R₁, R₂, R₃═H, R═CH₃)) (97.8 mg, yield of 77%). Mass spectrometry (ESI): literature value of 409.07, obtained value of 432.3 for [M+Na⁺]

### Production Example 3B

### Synthesis of Caged-NADP 2

Using 3,4-dimethoxy-2-nitrophenyl-acetophenone hydrazone, the target white powder (caged-NADP wherein the protecting group is represented by Formula I (R₁=4-methoxy, R₂=5-methoxy, R₃═H, R═CH₃)) was synthesized in the same manner as in Production Example 1B.

Mass spectrometry (ESI): m/z, literature value of 953.15 for [M⁺], obtained value of 953.2 for [M⁺]

### Production Example 4B

### Synthesis of Caged-G6P 2

Using 3,4-dimethoxy-2-nitrophenyl-acetophenone hydrazone, the target white powder (caged-G6P wherein the protecting group is represented by Formula I (R₁=4-methoxy, R₂=5-methoxy, R₃═H, R═CH₃)) was synthesized in the same manner as in Production Example 2B.

Mass spectrometry (ESI): m/z, literature value of 469.099 for [M], obtained values of 470.2 for [M+H⁺], 492.3 for [M+Na⁺], 508.1 for [M+K⁺], and 482.3 for [2M+Na⁺+H⁺]

### Reference Experiment 1B

### Photolysis of Caged-NADP

25 μL of aqueous solution of the caged-NADP in Production Example 1B before freeze-drying was placed in an Eppendorf tube and illuminated using a ultraviolet lamp (150 W, mercury-xenon lamp, manufactured by Hamamatsu Photonics K.K.) for minutes. The results showed that a peak at m/e=893.1 attributable to caged-NADP was reduced and the appearance of a peak at m/e=744.1([M+H⁺]) attributable to NADP was observed. This confirmed that a protecting group in caged-NADP can be decaged by UV light irradiation to give NADP.

### Production Example 5B

### Stable Expression of Human P450 Enzyme and P450 reductase in E. coli, and preparation of membrane fraction

### 1. Expression of Human P450 and P450 Reductase

Using a cassette plasmid for expressing P450, in which major human P450 (CYP1A1) and human NADPH-P450 reductase P450 were inserted in tandem with pCWRm1A2N, expression of P450 in E. coli was attempted. The transformation of E. coli was performed through the transformation of competent DH5α by a conventional method. Confirmation of the introduction of each plasmid into E. coli was conducted by evaluating the drug resistance by means of antibiotic ampicillin added to an LB medium. A culture of recombinant E. coli was initiated by inoculating a single E. coli colony on an LB agar medium that contained the antibiotic ampicillin to 2.5 mL of TB liquid medium. Pre-culturing was performed at 37° C. for 16 hours. Subsequently, culturing was performed in an LB medium containing an aminolevulinic acid having a final concentration of 500 μg/mL and ampicillin having a final concentration of 50 μg/mL for about 3 hours until the OD value became around 0.3. Upon lowering the temperature of the culture after culturing from 37° C. to 28° C., IPTG having a final concentration of 1 mM was added thereto and culturing was continued for 24 hours. The recombinant E. coli strains were collected from the E. coli liquid culture by centrifugation. The expression amount of each P450 enzyme protein in E. coli was evaluated by measurement using a reduced-CO difference spectrum. The reduced-CO difference spectrum was measured based on a conventional method by supplying CO under a reducing condition. The number of moles of P450 was calculated using the constant defined by Sato, Omura et al.

### 2. Purification of Membrane Fractions

E. coli membrane fractions were purified in the following manner. 200 mL of a TB culture medium was centrifuged at 3,000 g for 10 minutes to harvest. Thereafter, ultrasonic fragmentation was conducted 6 times each for 30 seconds to fragment the cells. Subsequently, a liquid containing the resulting cell fragments was centrifuged at 10,000 rpm for 10 minutes to separate residues in E. coli by centrifugation. The supernatant obtained after centrifugation was subjected to ultracentrifugation at 4° C. and 40,000 rpm (100,000 g) to collect membrane fractions containing P450 enzyme protein. Thereafter, the E. coli membrane fractions were dispersed in 3 mL of P450 storage buffer solution (100 mM potassium phosphate buffer (pH 7.5) containing 20% glycerol).

### Test Example 1B

### Activity Measurement

Drug metabolic activities of human CYP1A1 in the prepared recombinant E. coli were analyzed using high-performance liquid chromatography (HPLC). As the enzyme substrate, 7-ethoxycumarin (7EC), which is a P450 model fluorogenic substrate, was used. The following two enzymatic reaction methods were studied. One method directly added a substrate to a recombinant E. coli strain in which a previously cultured P450 enzyme protein was expressed. The other method used E. coli membrane fractions obtained by purifying a recombinant E. coli strain, in which a P450 enzyme protein was expressed, using an ultracentrifugal method. The oxidation reaction of P450, in the case where a P450 expressed E. coli strain was used, was conducted by adding various enzyme substrates in such a manner that each had a final concentration of 0.1 mM, and then incubating them at 28° C. for 50 hours. In the metabolism experiment using E. coli membrane fractions in which P450 was expressed, NADPH with a final concentration of 0.2 mM was added to the reaction solution as a coenzyme. The HPLC analysis was conducted using the D7000 HPLC System (manufactured by Hitachi Ltd.) with a C18 reverse phase column (COSMOCIL (5C18-AR), manufactured by Nacalai Tesque Inc.), and employing a linear gradient elution method using an eluent of MeOH/H₂O (containing 0.85% phosphoric acid) with a ratio of 35:65 to 100:0.

### Test Example 2B

### Measurement of Enzymatic Activity of Cytochrome P450 Using Caged-NADP

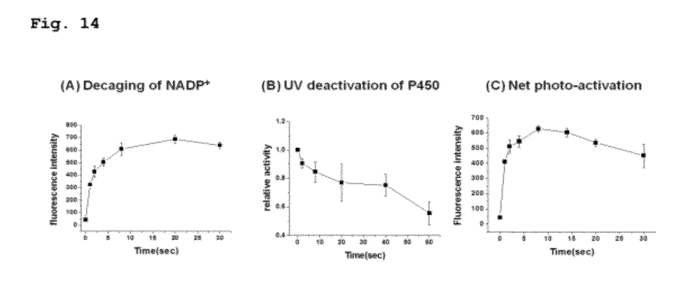

The enzymatic activity of cytochrome P450 was measured using caged-NADP. An aqueous solution obtained by mixing the following components was used as the reaction liquid: 50 μL of 1 M potassium phosphate buffer solution, 6.25 μL of 40 mM 7-ethoxyresorufin (7ER), 30 μL of 50 mM G6P, 2.89 μL of 69.3 U/mL glucose-6-phosphate reductase, 15 μL of 100 mM magnesium chloride,
1 μL of 5 mM caged-NADP aqueous solution, 10.25 μL of P450 membrane fractions (human CYP1A1), 5 μL of 0.1 M dithiothreitol, and 379.61 μL of ultrapure water. UV light irradiation (Ushio Spot Cure: Light intensity of 14 to 15 mW/cm² (365 nm)) was performed for different periods of time to transform caged-NADP to NADP, followed by incubation for 30 minutes to conduct a P450 enzymatic reaction. Thereafter, 25 μL of 30% trichloroacetic acid was added thereto to terminate the enzymatic reaction. By adding 500 μL of chloroform to the reaction liquid and stirring the mixture for 1 minute, the 7-hydroxycoumarin (7HR) generated by the reaction was extracted in chloroform. After centrifuging for 1 minute, 250 μL of the chloroform phase, which was the lower phase, was collected. By adding 500 μL of 0.01 M NaOH/0.1 M NaCl solution thereto and stirring the mixture for 1 minute, 7HR was re-extracted in the aqueous solution. After centrifuging for 1 minute, the upper phase was transferred to a cuvette, and the fluorescence spectra thereof were measured under the following conditions (Hitachi F-4500). Excitation wavelength: 366 nm, fluorescence wavelength: 380 to 600 nm. The maximum fluorescence was used to quantify the 7HR. To analyze the effect of decaging the caged-NADP by UV illumination, and the effect of deactivating the cytochrome P450 individually, measurements were also conducted under the following conditions. (A) Only caged-NADP was irradiated with UV light and added to the reaction liquid. (B) The entire reaction liquid, including P450, was irradiated with UV light and normal NADP was added thereto. FIG. 14 shows the results. In the case (A) where UV light irradiation was performed only on caged-NADP and the result was used for P450 activity assay, the P450 activity increased as the UV light irradiation increased, and it reached a fixed value in about 8 seconds. However, as shown in (B), a side effect was also found in which the activity of the P450 enzyme gradually decreased with UV light irradiation. This is probably because P450 is a hemoprotein containing a pigment. Accordingly, it was found that when caged-NADP was irradiated with UV light under the presence of a P450 enzyme, the dependence of P450 activation on the UV light irradiation time resulted in a behavior that summed the effects of decaging the caged-NADP and deactivating the P450 (C). It became clear that the optimal irradiation time for enzyme activation was about 8 seconds. It was also confirmed that a slight background reaction proceeded even with NADP in a caged condition because of the endogenous NADP contained in the P450 sample.

### Test Example 3B

### Measurement of Enzymatic Activity of Cytochrome P450 Using Caged-G6P

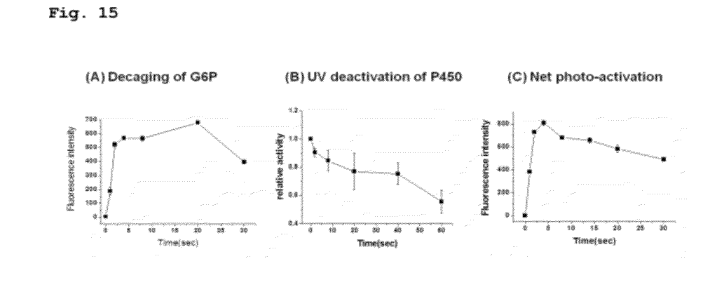

The enzymatic activity of cytochrome P450 was measured using caged-G6P. An aqueous solution obtained by mixing the following components was used as the reaction liquid: 50 μL of 1 M potassium phosphate buffer solution, 6.25 μL of 40 mM 7-ethoxyresorufin (7ER), 30 μL of 5 mM caged-G6P, 2.89 μL of 69.3 U/mL glucose-6-phosphate reductase, 15 μL of 100 mM magnesium chloride, 1 μL of 5 mM NADP aqueous solution, 10.25 μL of P450 membrane fractions (human CYP1A1), 5 μL of 0.1 M dithiothreitol, and 379.61 μL of ultrapure water. UV light was irradiated for different periods of time to transform the caged-G6P to G6P, followed by incubation for 30 minutes for a P450 enzymatic reaction. Thereafter, 25 μL of 30% trichloroacetic acid was added thereto to terminate the enzymatic reaction. By adding 500 μL of chloroform to the reaction liquid and stirring the mixture for 1 minute, the 7-hydroxycoumarin (7HR) generated by the reaction was extracted in chloroform. After centrifuging for 1 minute, 250 μL of the chloroform phase, which was the lower phase, was collected. By adding 500 μL of 0.01 M NaOH/0.1 M NaCl solution thereto and stirring the mixture for 1 minute, 7HR was re-extracted in the aqueous solution. After centrifuging for 1 minute, the upper phase was transferred to a cuvette, and the fluorescence spectra thereof were measured under the following conditions. Excitation wavelength: 366 nm, fluorescence wavelength: 380 to 600 nm. The maximum fluorescence was used to quantify the 7HR. To analyze the effect of decaging the caged-G6P by UV illumination and the effect of deactivating cytochrome P450 individually, measurements were also conducted under the following conditions. (A) Only caged-G6P was irradiated with UV light and added to the reaction liquid. (B) The entire reaction liquid, including P450, was irradiated with UV light and normal G6P was added thereto. FIG. 15 shows the results. In the case (A) where UV light was irradiated only on caged-G6P and the result was used for P450 activity assay, the P450 activity increased as the UV light irradiation increased and it reached a fixed value in about 4 seconds. However, a side effect was also found in which the activity of the P450 enzyme gradually decreased with UV light irradiation (B). Accordingly, it was found that when caged-G6P was irradiated with UV light under the presence of a P450 enzyme, the dependence of P450 activation on the UV light irradiation time resulted in a behavior that summed the effects of decaging the caged-G6P and deactivating the P450 (C). It became clear that the optimal irradiation time for enzyme activation was about 4 seconds. While in the case of caged-NADP, a slight background reaction proceeds even with NADP in a caged condition because of the endogenous NADP contained in the P450 sample, the background reaction for G6P under the caged condition is negligible. Therefore, when used alone, caged-G6P provides more precise photoregulation.

### Test Example 4B

### Measurement of Enzymatic Activity of Cytochrome P450 Using Caged-NADP and Caged-G6P in Combination

The results of Test Examples 2B and 3B indicate that both caged-NADP and caged-G6P are capable of regulating the enzymatic activity of P450 with a relatively short UV illumination time. When caged-NADP is used, a slight background reaction proceeds because of the endogenous NADP contained in the P450 sample. Therefore, the combined use of caged-NADP and caged-G6P enables stronger P450 activity regulation and more accurate enzymatic activity measurement.

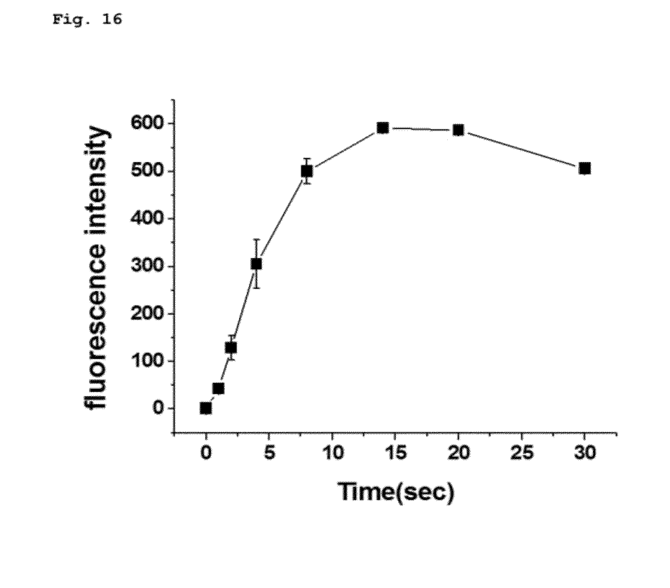

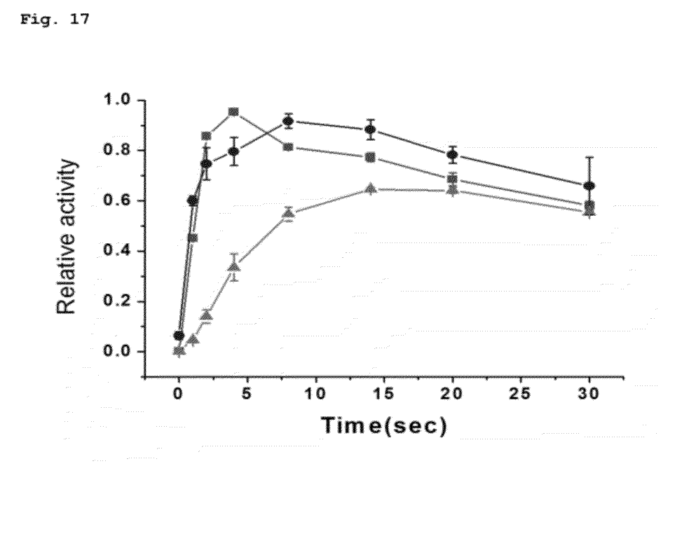

The enzymatic activity of cytochrome P450 was measured using caged-NADP and caged-G6P at the same time. An aqueous solution obtained by mixing the following components was used as the reaction liquid: 50 μL of 1 M potassium phosphate buffer solution, 6.25 μL of 40 mM 7-ethoxyresorufin (7ER), 30 μL of 5 mM caged-G6P, 2.89 μL of 69.3 U/mL glucose-6-phosphate reductase, 15 μL of 100 mM magnesium chloride, 1 μL of 5 mM caged-NADP aqueous solution, 10.25 μL of P450 membrane fractions (human CYP1A1), 5 μL of 0.1 M dithiothreitol, and 379.61 μL of ultrapure water. UV light irradiation was performed for different periods of time to transform caged-NADP and caged-G6P to NADP and G6P, respectively, followed by incubation for 30 minutes to conduct a P450 enzymatic reaction. Thereafter, 25 μL of 30% trichloroacetic acid was added thereto to terminate the enzymatic reaction. By adding 500 μL of chloroform to the reaction liquid and stirring the mixture for 1 minute, the 7-hydroxycoumarin (7HR) generated by the reaction was extracted in chloroform. After centrifuging for 1 minute, 250 μL of the chloroform phase, which was the lower phase, was collected. By adding 500 μL of 0.01 M NaOH/0.1 M NaCl solution thereto and stirring the mixture for 1 minute, 7HR was re-extracted in the aqueous solution. After centrifuging for 1 minute, the upper phase was transferred to a cuvette, and the fluorescence spectra thereof were measured under the following conditions. Excitation wavelength: 366 nm, fluorescence wavelength: 380 to 600 nm. The maximum fluorescence was used to quantify the 7HR. FIG. 16 shows the results. Compared to the case where caged-NADP or caged-G6P was used alone, longer UV light irradiation was required and the optimal irradiation time was about 15 seconds. It was also found that a P450 enzymatic reaction without UV light irradiation (a reaction using caged-NADP and caged-G6P) was smaller than one in which caged-G6P was used alone, thus enabling stricter regulation of P450 activity. FIG. 17 shows a summary of the results when caged-NADP or caged-G6P was used alone and when they were used in combination. The P450 activity was normalized by using the activity when normal NADP and G6P were used as a reference value. It can be seen that when caged-G6P was used alone, the activity became the maximum with the shortest UV light irradiation time, and the maximum activity value was larger than those measured under other conditions. On the other hand, when caged-NADP and caged-G6P were used in combination, a relatively long UV light irradiation time was required to activate P450 and the lowest activity was exhibited at the maximum value. These results show that the use of two types of caged compounds is advantageous for strongly suppressing activity under a caged condition. However, for the purpose of light activation, the use of caged-G6P alone can be considered to be the most effective.

### Test Example 5B

### Measurement of Enzymatic Activity of Cytochrome P450 Using Microwells

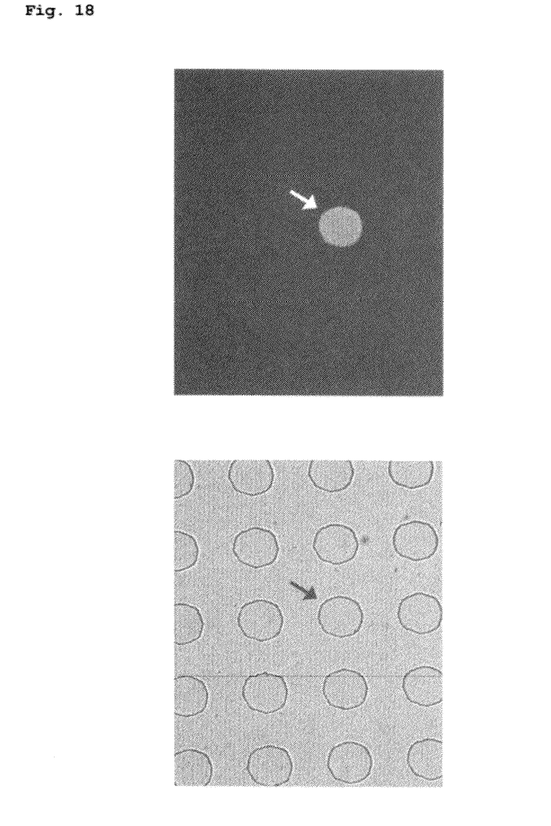

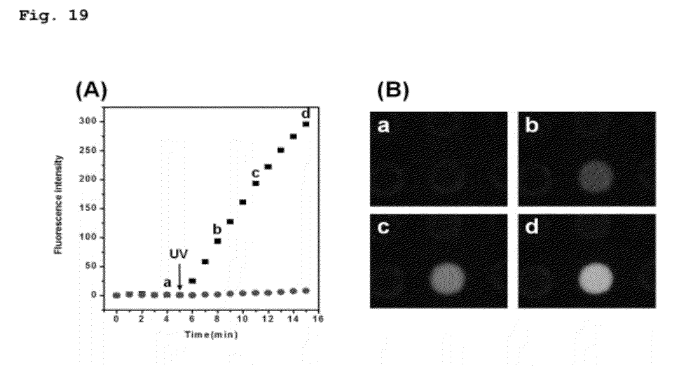

Using a caged compound allows enzymatic activity to be spatially controlled by localized UV light irradiation. To prove this, the following experiment was conducted. Microwells each having a width of 100 μm and a depth of 30 μm were produced using a silicone elastomer (polydimethylsiloxane: PDMS), and a reaction liquid for measuring cytochrome P450 enzymatic activity was introduced into the microwells. While observing with an optical microscope, the P450 enzyme was activated by locally irradiating UV light to activate P450 only in the irradiated microwell. An aqueous solution obtained by mixing the following components was used as the reaction liquid: 50 μL of 1 M potassium phosphate buffer solution, 6.25 μL of 40 mM 7-ethoxyresorufin (7ER), 30 μL of 5 mM caged-G6P, 2.89 μL of 69.3 U/mL glucose-6-phosphate reductase, 15 μL of 100 mM magnesium chloride, 1 μL of 5 mM NADP aqueous solution, 10.25 μL of P450 membrane fractions (human CYP1A1), 5 μL of 0.1 M dithiothreitol, and 379.61 μL of ultrapure water. The reaction liquid was encapsulated into each PDMS microwell by applying the reaction liquid to the surface of the PDMS microwells dropwise and sealing the wells with a glass slide. After observing the fluorescence in the microwells using a fluorescence microscope (BX51WI, Olympus Corporation) for 5 minutes (excitation wavelength: 545 to 580 nm, fluorescence wavelength: 610 nm or greater), the caged-G6P in the microwell was decaged by changing the wavelength of the excitation filter to 330 to 385 nm and irradiating the microwell for 8 seconds. The UV light irradiation region was limited to the single microwell by using a pinhole. Thereafter, the wavelength range of the excitation light was changed again and the observation was continued for 10 seconds. As a result, the cytochrome P450 became enzymatically active only in the irradiated microwell and fluorescence due to the metabolism of 7ER was observed (FIG. 18). While bright field microscope observation showed that the microwells were arranged at intervals of about 100 μm, fluorescence microscope observation revealed 7HR fluorescence in only the one microwell. FIG. 19 plots the fluorescence intensity in the microwell before and after UV light irradiation. The fluorescence intensity increased remarkably in the microwell irradiated with UV light; however, no increase in fluorescence intensity was observed in the neighboring microwells with intervals of about 100 μm. This experiment showed that the use of caged-G6P allows P450 activity to be controlled in a minute space.

### Test Example 6B

### Measurement of Enzymatic Activity of Cytochrome P450 Using Microwells

By combining a caged compound with a microarray or micro-flow channels, as shown in FIG. 12, the metabolic reactions of enzymes of multiple molecular species and multiple samples can be initiated at the same time. To prove this, the following experiment was conducted. Substrates (7ER) with different concentrations were placed in micro-flow channels each having a width of 60 μm and a depth of 30 μm together with cytochrome P450 enzyme (human CYP1A1) and caged-G6P, and the samples were simultaneously irradiated with UV light to activate the cytochrome P450 enzyme. An aqueous solution obtained by mixing the following components was used as the reaction liquid: 50 μL of 1 M potassium phosphate buffer solution, 30 μL of 5 mM caged-G6P, 2.89 μL of 69.3 U/mL glucose-6-phosphate reductase, 15 μL of 100 mM magnesium chloride, 1 μL of 5 mM NADP aqueous solution, 10.25 μL of P450 membrane fractions (human CYP1A1), 5 μL of 0.1 M dithiothreitol, 379.61 μL of ultrapure water, and 7ER with different concentrations. In order to decage the caged-G6P, UV light was irradiated on the entire region of the flow channel chip using Ushio Spot Cure. As a result, cytochrome P450 became enzymatically active in all of the flow channels, and fluorescence due to metabolic activity corresponding to the 7-ER concentration was observed (FIG. 20). This result indicates that enzymatic reactions can be simultaneously started in solutions containing various P450 molecular species and compounds with different concentrations by using microarrays or micro-flow channels to arrange them in parallel, and irradiating them. Analyzing the initial process of synchronized reactions is expected to be useful for quantitatively assaying the metabolic activities of P450 toward various compounds.

### Test Example 7B

### Responses Toward Different Substrate Concentrations (1)

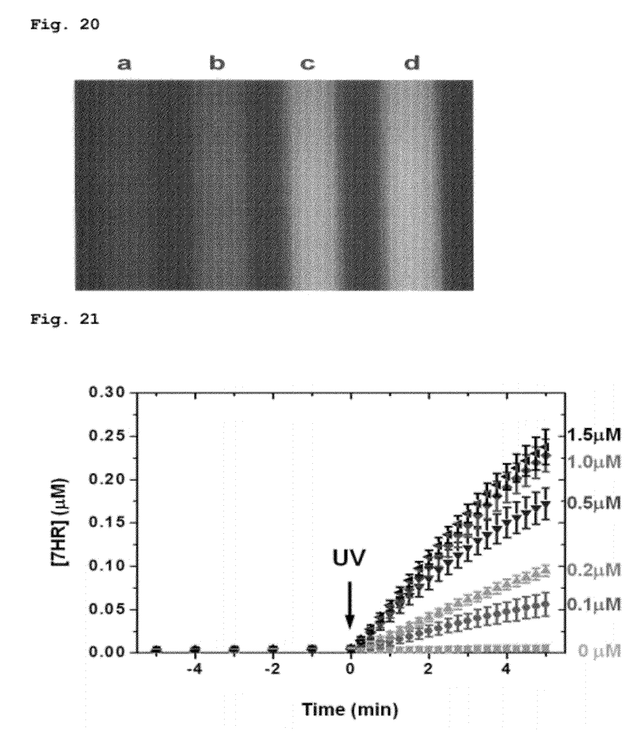

Using human CYP1A1 as P450 and 7ER as a substrate, the enzymatic reactions of P450 toward different substrate concentrations (0 μM, 0.1 μM, 0.2 μM, 0.5 μM, 1.0 μM, and 1.5 μM) were examined. Specifically, a PDMS slab having many microwells (width: 100 μm, depth: 30 μm) and a glass slide were laminated to encapsulate aqueous solutions each containing P450, a substrate, a coenzyme regenerating system (including caged-G6P), etc., inside microwells. When the caged-G6P was decaged by UV light irradiation under fluorescence microscope observation, the coenzyme (NADPH) that is necessary for P450 enzymatic activity was generated to start the enzymatic reaction, and increases in fluorescence corresponding to the substrate concentrations were observed (FIG. 21).

### Test Example 8B

### Responses Toward Different Substrate Concentrations (2)

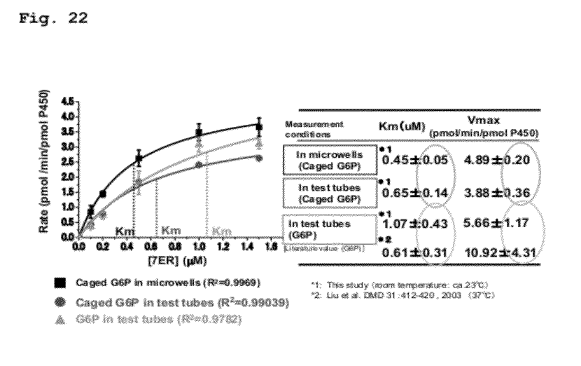

The results of measurement in metabolic activity toward different substrate (7-ER) concentrations were analyzed using Michaelis-Menten plots to determine the enzymatic kinetic constants (Kₘ, Vₘₐₓ) (FIG. 22). Michaelis-Menten plots (left) and reaction kinetic constants (right) of normal G6P were compared with those of caged-G6P. In the assay using normal G6P, a 2-mL test tube was used. The assay using caged-G6P was conducted in two ways, i.e., using a 2-mL test tube and using PDMS microwells. (In assays using caged-G6P, the reaction can be started with any desired timing by encapsulating solutions each containing an enzyme and a substrate in microwells. However, assays using normal G6P are difficult to conduct because the reaction starts while the solution is being mixed and encapsulated in the microwells.) Assays using caged-G6P exhibit smaller error values in Kₘ, Vₘₐₓ compared to those using normal G6P, enabling measurement with highly accurate data. The present invention allows the Kₘ, Vₘₐₓ of each enzyme to be measured in a highly accurate manner. It also enables valuable enzymes and substrate samples to be saved because the enzymatic reaction takes place in a miniscule space, such as a microwell.

### Test Example 9B

### Competitive Assay Using Fluorogenic Substrate

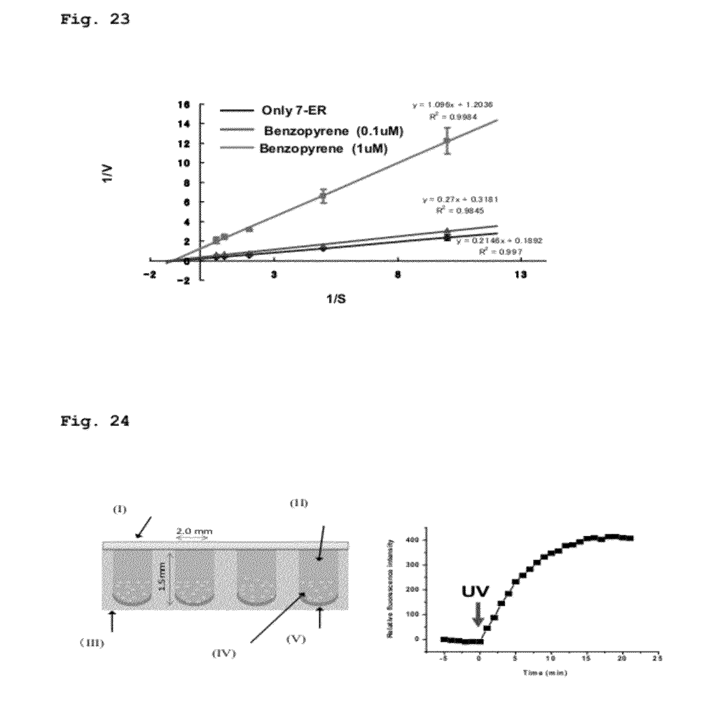

A competitive assay between a fluorogenic substrate (7-ER) and a non-fluorogenic substrate (benzopyrene) was conducted using caged-G6P. An aqueous solution obtained by mixing the following components was used as the reaction liquid: 50 μL of 1 M potassium phosphate buffer solution, 30 μL of 5 mM caged-G6P, 2.89 μL of 69.3 U/mL glucose-6-phosphate reductase, 15 μL of 100 mM magnesium chloride, 1 μL of 5 mM NADP aqueous solution, 10.25 μL of P450 membrane fractions (human CYP1A1), 5 μL of 0.1 M dithiothreitol, and 379.61 μL of ultrapure water. The 7-ER concentration was varied from 0.1 μM to 1.5 μM. The benzopyrene concentrations were 0.1 μM and 1 μM. After UV light irradiation, incubation was performed for 30 minutes to carry out the P450 enzymatic reaction. Thereafter, 25 μL of 30% trichloroacetic acid was added thereto to terminate the enzymatic reaction. By adding 500 μL of chloroform to the reaction liquid and stirring the mixture for 1 minute, the 7-hydroxycoumarin (7HR) generated by the reaction was extracted in chloroform. After centrifuging for 1 minute, 250 μL of the chloroform phase, which was the lower phase, was collected. By adding 500 μL of 0.01 M NaOH/0.1 M NaCl solution thereto and stirring the mixture for 1 minute, 7HR was re-extracted in the aqueous solution. After centrifuging for 1 minute, the upper phase was transferred to a cuvette, and the fluorescence spectra thereof were measured under the following conditions. Excitation wavelength: 366 nm, fluorescence wavelength: 380 to 600 nm. The maximum fluorescence was used to quantify the 7HR. FIG. 23 shows the results. FIG. 23 indicates the feasibility of a competitive assay using a fluorogenic substrate. When the effect of 7-ER on the initial reaction velocity was examined while changing the benzopyrene concentration, it became clear that benzopyrene acts on 7-ER as a noncompetitive inhibitor.

### Test Example 10B

### Detection of Enzymatic Activity Using Oxygen Sensor

It was shown that an enzymatic reaction could be started by encapsulating a reaction solution containing a fluorogenic substrate (7-ER), caged-G6P and other necessary reagents in microwells in which an oxygen sensor (ruthenium complex) and immobilized P450 (human CYP1A1)/agarose gel were vertically integrated, and irradiating the reaction solution with UV light (FIG. 24). As microwells, many wells each having a width of 2 mm and a depth of 1.5 mm were formed in a polymethylmethacrylate (PMMA) slab, and an oxygen sensing layer and an immobilized P450 (human CYP1A1)/agarose gel layer were vertically integrated (FIG. 24, left). Onto this vertically integrated chip, an aqueous solution (a mixture containing 50 μL of 1 M potassium phosphate buffer solution, 30 μL of 5 mM caged-G6P, 2.89 μL of 69.3 U/mL glucose-6-phosphate reductase, 15 μL of 100 mM magnesium chloride, 1 μL of 5 mM NADP aqueous solution, 10.25 μL of P450 membrane fractions (human CYP1A1), 5 μL of 0.1 M dithiothreitol, and 379.61 μL of ultrapure water) containing a substrate (capsaicin, 0.2 mM) was added, and the reaction solution was encapsulated using a sealing tape for microplates (FIG. 24, left). While observing the fluorescence of the oxygen sensing layer using a fluorescence microscope, the caged-G6P was decaged by using the light source of the microscope, and the oxygen consumption due to P450 enzymatic activity was observed as an increase in the fluorescence intensity (FIG. 24, right).

### INDUSTRIAL APPLICABILITY

The vertically integrated chip of the present invention, which comprises immobilized cytochrome P450 and an oxygen sensor, enables fast, high-sensitivity detection of the metabolic reactions of various P450 molecular species toward compounds. The technique of the present invention for photoregulating the enzymatic activity of P450 by using a caged compound makes it possible to evaluate the enzymatic activity of P450 in an accurate and highly efficient manner by measuring the initial reaction velocity of cytochrome P450 enzymes encapsulated in numerous miniscule spaces. The use of these techniques allows a comprehensive, efficient and accurate prediction of the type of P450 that will metabolize a certain compound and the approximate velocity of the metabolism. Therefore, these techniques are applicable to, for example, biotransformation systems utilizing P450 oxidation reactions; systems for evaluating compound conversion ability for drug development; systems for predicting the metabolic activity of compounds in vivo for drug development; food inspections; and safety evaluations for drugs and foods that reproduce human polymorphisms. Comprehensive detection of P450 enzymatic activity is also useful in the fields of test diagnosis, bioanalysis (analyzing the drug concentration in biological samples), and culture media and reagents for food sanitation inspections.

1-21. (canceled)

22. A vertically integrated chip comprising an oxygen sensing layer and a cytochrome P450-supporting layer vertically integrated on a chip,
in the cytochrome P450-supporting layer, cytochrome P450 being supported in a hydrophilic polymer matrix, wherein said hydrophilic polymer matrix is a matrix of a hydrophilic polymer,
the oxygen sensing layer comprising an oxygen sensor and a matrix,
said chip meets at least one requirements (1) to (4):
(1) the oxygen sensing layer and the cytochrome P450-supporting layer are vertically integrated in a micropore (microwell)
(2) said chip further comprises a flow channel for introducing a substrate onto the cytochrome P450-supporting layer, wherein an oxygen sensor and enzyme-immobilized gel are vertically integrated in the flow channel
(3) the oxygen sensing layer and the cytochrome P450-supporting layer are vertically integrated in the micro-flow channel in a uniform manner.
(4) said cytochrome P450-supporting layer is immobilized on the surface of an oxygen sensing layer which is formed on the chip.

23. The vertically integrated chip according to claim 22, wherein the hydrophilic polymer is agarose gel.

24. The vertically integrated chip according to claim 22, wherein the oxygen sensing layer contains a ruthenium complex in a silica matrix.

25. The vertically integrated chip according to any one of claims 22 to 24, wherein the flow channel is a micro-flow channel.

26. The vertically integrated chip according to claim 25, wherein the cytochrome P450-supporting layer comprises a plurality of cytochrome P450-supporting portions each having a cytochrome P450, and the vertically integrated chip is capable of analyzing metabolic activity of each cytochrome P450 toward a substrate.

27. Use of the vertically integrated chip of claim 26 to evaluate the degree of oxidation reaction of the substrate due to cytochrome P450.

28. A method for identifying a compound comprising:
reacting a substrate with the vertically integrated chip of claim 26 to identify the compound based on metabolic patterns of a plurality of cytochrome P450s and substrates.

29. Use of at least one caged compound selected from the group consisting of caged-NADP and caged glucose-6-phosphate (G6P) to evaluate the degree of oxidation reaction of the substrate due to cytochrome P450, wherein cytochrome P450 is supported in the cytochrome P450-supporting layer of the vertically integrated chip according to claim 26, wherein the caged-NADP is represented by the following formula: 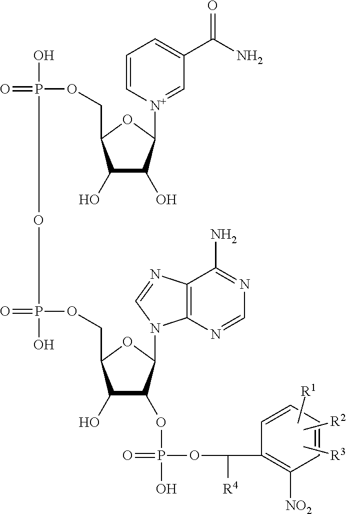
wherein R¹, R² and R³ may be the same or different and independently represent a hydrogen atom, a lower alkyl group, a lower alkoxy group, an amino group, a halogen atom, a hydroxy group or a cyano group; or any two of R¹, R² and R³ are combined to form a methylenedioxy group; and R⁴ represents a hydrogen atom or a methyl group, wherein the caged-G6P is represented by the following formula: 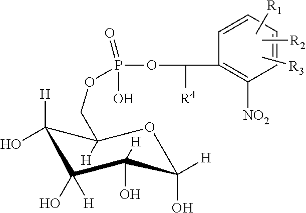
wherein R¹, R² and R³ may be the same or different and independently represent a hydrogen atom, a lower alkyl group, a lower alkoxy group, an amino group, a halogen atom, a hydroxy group or a cyano group; or any two of R¹, R² and R³ are combined to form a methylenedioxy group; and R⁴ represents a hydrogen atom or a methyl group.

30. A kit comprising at least one caged compound selected from the group consisting of caged-NADP and caged-G6P, a cytochrome P450 reductase and the vertically integrated chip according to claim 26, the kit being used for measuring the enzymatic activity of the cytochrome P450 toward a substrate compound.

31. The kit according to claim 30, which comprises both the caged-NADP and the caged-G6P.

32. The kit according to claim 31, which comprises a microwell structure or a micro-flow channel, and which simultaneously activates various types of cytochrome P450 reductases by locally or entirely irradiating light to measure the activities thereof in parallel.
